(19) United States
(12) Patent Application Publication
(10) Pub. No.: US 2026/0123877 A1
(43) Pub. Date: May 7, 2026

(54) Transmission Device

(71) Applicant: Andreas Froese, Steinbach (CA)

(72) Inventor: Andreas Froese, Steinbach (CA)

(21) Appl. No.: 18/703,620

(22) PCT Filed: Sep. 25, 2023
(86) PCT No.: PCT/CA2023/051236

§ 371 Date: Apr. 22, 2024

(30) Foreign Application Priority Data
Nov. 24, 2022 (CA) 3182385
Mar. 22, 2023 (CA) 3193780

(51) Int. Cl. A61B 5/00; A61B 5/055; G06T 7/00; G06T 7/11; G06T 7/62

(52) U.S. Cl. CPC A61B 5/4064; A61B 5/0042; A61B 5/055; A61B 5/4082; A61B 5/4088; A61B 5/4806; A61B 5/4842; G06T 7/0014; G06T 7/11; G06T 7/62; A61B 2576/026; G06T 2207/10088; G06T 2207/20081; G06T 2207/20112; G06T 2207/30016

(57) ABSTRACT

A method of diagnosing a neurodegenerative disorder (ND) in a patient comprising: (a) obtaining MRI image(s) of the patient's brain, (b) using the MRI image(s) of the patient's brain to segment sub-cortical structures associated with the ND into sub-regions, based on structural connectivity to cortical sub-regions, (c) extracting one or more MRI features from each of the sub-regions generated by the segmentation, and (d) using one or more machine learning techniques to classify the patient as being ND positive or ND negative based on comparisons of the one or more MRI features to at least one training data set that includes MRI features of each of the sub-regions generated by the segmentation of known ND positive controls and MRI features of each of the sub-regions generated by the segmentation of ND negative controls, thereby diagnosing ND. Also computer-based or cloud-based systems to diagnose a ND in a subject.

### FIELD OF TECHNOLOGY

This disclosure relates to diagnostic, progression, and prognostic tests of neurodegenerative disorders using magnetic resonance imaging (MRI). More particularly, this disclosure relates to the identification of MRI biomarkers that allow for the diagnosis, staging, sub-typing and prognosis of neurodegenerative disorders using MRI, automated imaging analysis, and machine learning.

### BACKGROUND INFORMATION

Neuronal loss and/or biochemical dysfunction in subregions of subcortical structures, such as the substantia nigra pars compacta (SNc) and the striatum, often underlie neurodegenerative diseases. For example, Parkinson's disease (PD) is a progressive neurodegenerative illness that is highly heterogeneous, with wide-ranging symptoms. PD patients manifest motor abnormalities such as rest tremor, slowed movements (i.e., bradykinesia) that are reduced in size and number (i.e., hypokinesia), and muscular rigidity.^{1,2} Currently, PD is diagnosed clinically when these cardinal motor symptoms appear, which arise due to degeneration of dopamine-producing neurons in the SNc and consequent dopamine restriction to subregions of the striatum. Dopamine-producing neurons in the ventral tegmental area (VTA), adjacent to the SNc and substantia nigra par reticulata (SNr), are only affected later in PD. Subcortical structures such as the SNc/VTA (substantia nigra pars compacta/ventral tegmental area) and the striatum, as well as the globus pallidus interna (GPi), globus pallidus externa (GPe), and the subthalamic nucleus (STN), are gray matter structures (i.e., clusters of neurons) located deep in the brain (FIG. 1). The SNc and VTA are adjacent subcortical structures, comprised of dopamine-producing neurons, that are not easily separated using standard approaches to imaging, though they are differentially affected by PD and are therefore important to measure separately. The striatum comprises the caudate nucleus, putamen, and nucleus accumbens (FIG. 1). The outer boundaries of subcortical structures, including the caudate nucleus and putamen, are easily discerned using neuroimaging such as MRI. However, subcortical structures lack internal margins to reliably delineate segments that are diverse in terms of function and vulnerability to diseases. This has limited the usefulness of neuroimaging to diagnose, track, and predict evolution of neurodegenerative disorders, which often involve abnormalities in subcortical structures.

PD patients also experience a wide range of other motor and non-motor symptoms such as sleep and mood disorders, cognitive deficits, and autonomic dysfunction. Pathophysiology of these other motor and non-motor symptoms is more poorly understood. There is significant heterogeneity across PD patients in a) the intricate array of motor and non-motor symptoms that they experience, b) the stage of disease when symptoms appear, as well as c) the severity of symptoms and rates of progression of PD. Taken together, PD is a complex disorder, involving wide-ranging brain regions, that is difficult to diagnose and manage.

There are currently no objective tests to diagnose PD in routine clinical practice (add reference here). PD is diagnosed through lengthy and complicated clinical assessments, ideally performed by neurologists with movement disorders expertise (i.e., the current diagnostic gold standard). Unfortunately, there are vastly insufficient numbers of movement disorder specialists. For example, there are approximately 80-100 movement disorder specialists practicing in Canada for a population of 38,781,291). In 2022, there were up to 90,000 people diagnosed with PD each year in North America, but there were as few as 88 movement disorders fellowship spots (i.e., specialist training opportunities) available each year in North America. Consequently, there were nearly 1,000 newly diagnosed PD patients alone for every newly-trained movement disorder neurologist. Movement disorder neurologists care not only for PD patients, but also for patients with other parkinsonian conditions and a diversity of other movement disorders. Currently, PD affects more than 3% of the population aged over 65. However, PD is the fastest-growing neurological disease in the world, due in part to its association with aging and ongoing demographic shifts and hence the gap between the availability of movement disorder specialists and PD patients will only increase. The 2011 prevalence of PD patients is predicted to double by 2031 in countries such as Canada. PD is a disabling disorder that has high economic costs. PD-related expenses exceed $50 billion annually at its current prevalence in the United States and significant expense is related to delayed diagnosis and introduction of symptomatic treatment.

Diagnostic judgments and medication titration are optimally performed by movement disorder specialists, but more commonly are achieved by clinicians with lesser degrees of PD expertise. Diagnostic ambiguity is common in PD, especially at early stages of disease, and treatment is often delayed while diagnoses are clarified. Wait times for specialist assessments can be substantial and levels of function can deteriorate significantly during this time, occasionally provoking changes in life circumstances (e.g., early retirement, relocation), though dopaminergic medication, dosed proficiently, can cause significant and rapid improvements. Patients with atypical PD features or with PD mimics (i.e., diseases that overlap in some symptoms with PD, but differ in their underlying pathophysiology) can require many clinical visits and second opinions before a clear diagnosis is achieved. Many patients with complex presentations of PD or PD mimics, who do not have access to experienced movement disorder clinicians, never receive satisfactory diagnoses or treatment. This not only adversely impacts patients themselves but also their families who grapple with questions about the heritable potential of the undiagnosed condition of a family member.

The want of accurate, reliable, and generally accessible biomarkers, in the form of objective diagnostic, progression, or prognostic tests to identify patients with PD, from healthy controls and patients with symptoms that appear clinically similar to PD (i.e., PD mimics), causes inefficiency and uncertainty in current clinical management as well. The PD mimics include patients who endorse symptoms and/or exhibit signs of Parkinsonism (e.g., tremor, bradykinesia, rigidity, gait impairment) but do not have PD. These PD mimics include Multiple Systems Atrophy (MSA), Progressive Supranuclear Palsy (PSP). Corticobasal Ganglionic Degeneration Syndrome (CBS), Essential Tremor, Lewy Body Dementia (LBD), or sub-types of Neurodegeneration with Brain Iron Accumulation (NBIA). PD is diagnosed, and dopamine-replacement therapy is introduced and adjusted, through time-consuming clinical assessments.

Accurate, objective, and easily accessible diagnostic tests would transform PD management immediately. These tests would empower generalists with less experience in PD (e.g., family physicians, internists) to diagnose and treat early staged or less complex PD patients with greater accuracy, confidence, and promptness. This would free up specialists to treat later staged and more complicated PD patients. Furthermore, these measures would avoid referrals of patients with common, benign PD mimics (e.g., Essential tremor) to movement disorder neurologists for the purpose of ruling out PD. In contrast, for patients with much rarer, malignant PD mimics (e.g., PD plus conditions: MSA, PSP, and CBS), definitive diagnostic tests of PD versus PSP, MSA, or CBS would a) eliminate substantial periods of diagnostic uncertainty—a highly consequential development given significantly reduced life expectancy for patients with PD plus conditions, and b) ensure PD plus patients are prioritized for speedy assessment by movement disorder specialists who can explain diagnoses and prognoses, provide support for serious neurological impairments, and assist in making end-of-life decisions. In this way, care of patients with parkinsonism would immediately be improved by the proposed innovation, beyond increasing the potential for uncovering disease-modifying therapies of PD or PD plus conditions, which is also an urgent unmet need. Appropriate triage of patients with parkinsonian conditions—an immediate result of the proposed innovation, is one of the few conceivable solutions that is achievable in the near-term, toward bridging the imminently-enlarging gap between needs of patients with neurodegenerative conditions and available services, considering the projected sharp rise in prevalence of aging-associated illnesses such as PD, parkinsonian conditions, and most other neurodegenerative diseases (e.g., Alzheimer's disease).

There are no cures or disease-modifying therapies for PD. Dopamine replacement medications temporarily alleviate movement symptoms without affecting PD progression (i.e., symptomatic treatments). They are highly effective in improving motor deficits at early stages of disease. However, at later stages, high doses of dopaminergic therapy are needed to keep pace with disease progression, causing disabling side effects. Furthermore, some motor (e.g., freezing of gait) and most non-motor symptoms of PD are not dopamine-responsive at any stage of disease. Therapies for these other symptoms have limited efficacy.

The lack of accurate, reproducible, as well as broadly available and easily-administered measures to identify PD and track its progression is an obstacle to developing interventions that truly alter PD advance. In the absence of an exact diagnostic test that can be easily applied, PD patients are enrolled in clinical trials at later stages of disease, when the clinical presentation is clearer. However, therapies are predicted to have greatest disease-modifying potential when they are introduced in the earliest stages of neurodegenerative diseases or, where possible, in pre-clinical or prodromal stages of disease. Furthermore, patients who do not have PD but who exhibit symptoms that overlap with those of PD (i.e., PD mimics) are currently unavoidably included in clinical trials, reducing power to detect truly disease-modifying effects. Finally, reliance on insensitive and/or variable and inconsistent clinical endpoints (e.g., self-report questionnaires, physical exams performed by clinicians who vary in expertise), rather than reproducible, quantifiable measures (i.e., objective progression biomarkers) can obscure beneficial impacts of interventions. This is even while these trials currently are costlier and require larger numbers of participants who are followed for longer, to increase the chance of clearly establishing benefit of an intervention, if indeed it helps. Finally, measures that could identify PD patients, at baseline, who are likely to progress faster than others, or to develop certain late-stage symptoms (e.g., freezing of gait, or cognitive impairment), could be useful in improving the efficiency and power of clinical trials, some of which might target specific symptoms.

The current gold standard for diagnosing PD is clinical diagnosis by movement disorder neurologists. Rizzo and colleagues⁴ performed a meta-analysis of the diagnostic accuracy of PD, reviewing 20 studies and more than 7000 patients, including 11 studies that referred to autopsy data. They found that PD diagnostic accuracy was 83.9% for movement disorder specialists who followed patients over years. More often, the diagnosis is performed by clinicians with lesser expertise such as general neurologists, geriatricians, internists, or family doctors, with average accuracies of 73.8% for patients followed over years.⁴ On the higher end of the spectrum, in a more recent study performed at the National Hospital for Neurology and Neurosurgery (Queen Square, UK), Virameteekul and colleagues⁵ reported a diagnostic accuracy of 91.5% for movement disorder neurologists and of 84.2% for neurologists and geriatricians, in patients who were seen and followed within five years of symptom onset. When clinicians rely on the International Parkinson's Disease and Movement Disorders Society (MDS) PD Diagnostic Criteria, accuracies improve. The MDS PD Diagnostic Criteria consists of a set of complex inclusion and exclusion criteria, supportive features, and ‘red flags’. The application of these criteria requires broad and deep knowledge of neurological diseases, particularly movement disorders, and therefore the application of these criteria by generalists is extremely challenging.

### PD Biomarkers: Ongoing Investigations

Recently, seeding amplification assays for the detection of misfolded α-synuclein in the cerebrospinal fluid (CSF) have shown potential to identify PD patients or patients with PD prodromal conditions such as rapid eye movement (REM) behaviour sleep disorders (RBD). The latter have a high rate of conversion to conditions such as PD and related Parkinsonian conditions (i.e., MSA and Lewy Body Dementia) that are characterized by misfolded a-synuclein, forming Lewy bodies (i.e., α-synucleinopathies). Siderowf et al⁶ found that the α-synuclein seed amplification assay identified PD patients (n=545) within 2 years of diagnosis from healthy individuals (n=163) with a sensitivity of 87.7% and a specificity of 96.3%. This assay also identified prodromal patients (n=51) with 86% sensitivity. A more recent study of very early-staged PD (n=121) distinguished PD patients from healthy controls (n=51) with sensitivity of 82.6% and specificity of 88.2%. 7

CSF α-synuclein seeding amplification assays show promise to potentially distinguish PD patients from a) healthy individuals and b) those PD mimics not associated with abnormal α-synuclein accumulations in the form of Lewy Bodies (e.g., PSP, CBS). However, with this method, distinguishing PD patients from PD mimics in whom abnormal α-synuclein accumulates (i.e., α-synucleinopathies), such as MSA and Lewy Body Dementia. There is little potential in this technique for differentiating PD sub-type. Furthermore, this is a qualitative assay (i.e., + or −) therefore has no ability to measure disease progression, severity. Given that this produces a single measure only, there seems to be no potential for sub-typing PD patients into clinical sub-groups based on symptoms or disease severity. Finally, CSF samples are obtained through lumbar punctures which is an invasive procedure that carries some risks and discomfort. This procedure is only performed by practitioners with some expertise.⁶

Neuroimaging has revolutionized management of most neurological diseases. MRI, due to its ubiquity, non-invasiveness, safety, as well as ever-increasing resolution of images and sophistication of analyses has enormously improved research and practice of most neurological diseases. As a few examples, MRI has come to play a central role in clinical trials evaluating the efficacy of disease-modifying medications in multiple sclerosis (MS), in the diagnosis of stroke, CNS infections, in the evaluation of aetiology of epilepsy, as well as in tracking progression of brain tumours and Huntington's disease.

In contrast, PD is the lone, common neurological illness of the CNS in which neuroimaging plays little role. DaTscan permits presynaptic dopamine transporter measurement in striatum, using a radioactive tracer (i.e., [123I]Ioflupane) and single positron emission computed tomography (SPECT). Though it has been available for over a decade and in theory seems an ideal measure for identifying PD and monitoring its progression, recent reviews of this literature confirm that it is not widely used in clinical research or practice, especially outside of specialist centres. This is due to problems with specificity in early stages of PD (i.e., some PD mimics also have positive DaTscans), insensitivity to disease progression, scarce accessibility, and concerns about radiation exposure, particularly for serial testing.

An abundant literature has accumulated over the last two decades, investigating positron emission tomography (PET), as well as structural and functional MRI approaches toward accurately and consistently diagnosing and tracking PD. Despite some promising findings, most studies have investigated only group-level differences (see Table 1). The minority of studies in this literature have sought to perform single-subject level distinctions of PD patients from healthy age-matched controls (see Table 1, Classification Model) or from patient control groups (e.g., PD mimics, other diseases such as Alzheimer's; see Table 1, Classification Model), though the purpose of diagnostic tests is to inform individual patients about their clinical conditions. Studies that have sought to distinguish PD patients from healthy controls, the most technically challenging discrimination, are scarce, particularly in light of the enormity of the literature investigating PD with neuroimaging approaches. Ultimately, to this point, there are no biomarkers that are objective, reproducible, and practical/useful for diagnosis, prognosis, or staging of PD (see Tables 1 and 2).

Recent investigations of MRI, coupled with machine learning and artificial intelligence techniques have suggested potential to classify PD patients from healthy controls at the individual level (Table 2). Some studies demonstrate high classification accuracies that would seem sufficient to translate to clinical contexts. Nonetheless, none have emerged to fulfill this role. A critical review of this literature, however, reveals several weaknesses that potentially explain this failure to progress to practical implementation. Small sample sizes (e.g., n<100) in a patient population as heterogeneous as PD, raise concerns that results in these studies will not reproduce, nor generalize. Referring to Table 2, only six of the most promising studies in this literature test PD groups larger than 100. These small sample sizes hamper the stability and generalizability of machine learning models. Related to the issue of generalizability, most studies in this literature do not test the classification models that they develop in an independent test set. Only six of the most promising studies include an independent test set and an additional four use cross-validation or leave-one-out analyses to improve the generalizability of their models. Along similar lines, details outlining the rigour of the machine learning approach are often lacking. Repeated random splits of the data, randomizing starting seeds, performing multiple replications and reporting average ROC/AUC, sensitivities/specificity rather than the highest/best results (Table 2), would inspire greater confidence in these approaches and could lead to advances. The AUC (area under the curve) value is a measure of accuracy. It is a function of sensitivity and specificity. Depending on how the dataset is split into training and test sets, the calculated AUC can fluctuate, especially with smaller datasets. Only three of the most promising studies in Table 2 present their average AUC values from multiple experiments. Another issue that inflates the promise/power of previous investigations into the potential of neuroimaging to diagnose, track, and prognose PD, is the reporting of combined accuracies of neuroimaging approaches and clinical measures or expert rating scales (Table 2). Given that a principal aim of developing widespread biomarkers of PD/ND is to alleviate the burden on medical specialists, this tactic undermines these approaches. Finally, to truly translate these findings to clinic or clinical research, having automated imaging analysis approaches seems critical. Methodologies that require significant neuroradiological imaging experience or computational expertise are limited in their potential for clinical translation from the start. Only a few studies automate components of their approaches, and none to date automate these complex computational algorithms from end-to-end as would be required to truly translate.

Measures of the N1 nigrosome of the SNc (the first and most affected region in early PD) are the most promising approaches to date (Table 2; Aziz et al, Jokar et al.). These measures have replicated across labs and the elements of this technique are now automated. However, the current studies include few patients (all less than 100 per group) and it is known that a proportion of PD patients fail to show imaging abnormalities in these regions even when expert neuroradiologists. For example, a recent study revealed only 37% sensitivity and 78% specificity for diagnosing PD from healthy controls.⁸ Conversely, these abnormalities are found frequently in PD mimics and healthy controls (eg. 63% abnormalities in this region in patients with Lewy Body Dementia⁹; MSA and healthy controls¹⁰). Only studies with ample sample sizes, far beyond hundreds, can truly investigate these issues. Finally, this measure has limited or no potential to measure progression of PD given that the N1 nigrosome of the SNc has degenerated to significant degree by the time PD appears, in those patients who will exhibit this sign.¹⁰

To date, previous approaches have segmented the SNc as detailed in the paragraph above and the subthalamic nucleus (STN) for improving the targeting for lead placement of deep brain stimulators in those patients who require this form of therapy. Previous studies have not segmented subcortical regions into subregions beyond the external boundaries that are visible on MRI, in a principled and reproducible way, toward extracting measures for diagnostic, progression, or prognostic purposes. In particular, no previous studies have segmented the striatum into subregions beyond its components that are visibly separable on neuroimaging (i.e. the caudate nucleus and the putamen). This is despite the fact that the striatum is highly heterogeneous in its function and its susceptibility to disease, with at least seven functional subregions as per meta-analyses.¹¹ The striatum is a central structure in PD and other NDs.

Clear external borders give the false impression that subcortical structures are uniform and homogenous. Similar to other gray structures, such as the cerebral cortex, however, subregions of subcortical structures differ in structure, function, as well as in their susceptibility to diseases, such as PD. Measures of whole subcortical structures are convenient but are anticipated to be insensitive markers of disease because they combine estimates across heterogeneous regions. For example, the pathophysiology of PD motor symptoms is fortunately well understood. They arise when a sufficient number of dopamine-producing neurons in the caudal motor segment (i.e., the dorsolateral portion; N1 nigrosome) of the SNc are lost, causing severe dopamine restriction to, and secondary degeneration of, the caudal motor subregion of the striatum (i.e., dorsolateral portions of both the caudate nucleus and the putamen). Despite these clear pathophysiological changes in the SNc and striatum, neuroimaging measures of the total SNc and/or striatum do not consistently differ between PD patients and healthy controls, even at the group level, let alone with sufficient sensitivity to detect patients at the single-subject level—the aim of a biomarker. The caudal motor subregions of the SNc and striatum represent very modest portions of the total SNc and striatum (FIG. 1), likely obscuring any potential effects.

In contrast to subcortical structures, however, the cortex is divided into distinct subregions due to gyri and sulci that act as visible landmarks, as well as validated MRI atlases/coordinates (FIG. 1). MRI measures targeting brain regions linked to bradykinesia/hypokinesia and rigidity—the only symptoms that occur in every PD patient—are predicted to have greatest potential to contribute to a sensitive, specific, and consistent diagnostic marker. However, the ability to obtain targeted or isolated measures of distinct subcortical subregions, is thwarted by the lack of visible or detectable internal boundaries on neuroimaging.

The proposed innovation capitalizes on the topographical organization of the brain (i.e., adjacent neurons/areas in one region, project to adjacent neurons/areas in another) and the objective definition of cortical subregions using publicly available MRI atlases. Using tractography, a process that estimates pathways between distant brain regions (e.g., cortex and striatum, striatum and SNc/VTA) by integrating voxel-wise fibre orientations, the innovation seeks to segment subcortical structures into subregions, allowing more targeted measures of distinct subcortical segments that are sensitive to neurodegenerative diseases (FIG. 1). The proposed innovation includes an analysis pipeline that performs automated, subject-specific segmentation of subcortical structures, such as the striatum and SNc/VTA, based on connectivity to cortical subregions. Cortical subregions are specified using coordinates from publicly available atlases. As an example of this methodology, based on Tziortzi and colleagues' meta-analysis of functional neuroimaging studies of the striatum, we segmented the striatum into seven subregions using our pipeline. First, the cortex was partitioned into seven regions using explicit coordinates from the public Harvard-Oxford neuroimaging atlas. Next, using the CIT168 atlas (reference), the total striatum, was outlined objectively. This ensures that every step of the segmentation process is objectively specified, entirely reproducible, and not dependent upon expertise of users (e.g., accurate detection and/or manual tracing of structures). Next, using probabilistic tractography—a method for tracing white matter tracts (see Connectivity-based parcellation of the striatum: Volume and connectivity measures, for further details), the striatum was partitioned into seven segments, dictated by the cortical region to which each striatal voxel predominantly connected (FIG. 2). This segmentation enabled targeted measures of striatal subregions that were predicted to be most sensitive to PD. As a proof of principle, we found that the volume of the caudal motor striatum, the region first and most dopamine depleted in PD, was reduced for PD patients compared to age-matched healthy controls, at the group level. Consistent with the larger literature, the volume of the total striatum, caudate nucleus, and putamen were statistically equivalent between the groups of PD patients and healthy age-matched controls.

Once subcortical segments are formed based on connectivity to cortical subregions, measures of volume, shape, iron, connectivity/related white matter tracts, and blood oxygenation-level-dependent (BOLD) signals are extracted from each subcortical subregion.

Furthermore, using a convolutional neural network (CNN) approach, disease-, symptom-, and/or progression-relevant features from each subcortical segment will be detected through training. CNN can learn to identify relevant features through training, allowing extraction of information from the image segments directly, recognizing patterns and features that are not anticipated in advance. Once features are extracted, they are combined in machine learning analyses, such as random forest classification, neural networks, Bayesian machine learning, to achieve models that will identify patients with neurodegenerative illnesses, preclinical neurodegenerative illnesses, sub-types of neurodegenerative diseases. These features will also be combined to detect progression and to predict symptom/sub-type development, disease progression/severity. These models are incorporated into our automated image analysis, image segmentation, and patient classification/staging/prognostication pipeline, to enable diagnostic, staging, and prognostic decisions. In this way, our procedure is automated end-to-end, requiring no specialized expertise or domain knowledge, providing results at the individual level, based on neuroimaging features using structural, diffusion, iron, and functional MRI.

### SUMMARY OF DISCLOSURE

In one embodiment, the present disclosure is a method of diagnosing a neurodegenerative disorder (ND) in a patient, the method comprising: (a) obtaining one or more magnetic resonance imaging (MRI) images of the patient's brain, (b) using the one or more MRI images of the patient's brain to segment one or more sub-cortical structures associated with the ND into sub-regions, based on structural connectivity to cortical sub-regions, (c) extracting one or more MRI features from each of the sub-regions generated by the segmentation in part (b) of the patient's brain, and (d) using one or more machine learning techniques to classify the patient as being ND positive or ND negative based on comparisons of the one or more MRI features to at least one training data set, the at least one training data set including MRI features of each of the sub-regions generated by the segmentation of known ND positive controls and MRI features of each of the sub-regions, generated by the segmentation of ND negative controls, thereby diagnosing ND in the patient. In aspects, the cortical sub-regions are defined using a public MRI atlas. In another aspect the one or more MRI features are compared to one or more models developed using the at least one training data set. In another aspect, the one or more features are compared to (a) one or more models developed using the at least one training data set and (b) the at least one training data set. In another aspect, the one or more MRI images of the patient's brain is one or more MRI images of the patient's cortex.

In one embodiment of the method of diagnosing a ND in a patient, the one or more MRI features include measures of surface area, surface displacement [relative to average shape of age-matched HC group], volume, connectivity/related white matter tracts, and/or quantitative MRI parameters, the latter enabling estimation of heavy metals, such as iron, BOLD signal, and image features developed through convolutional neural network (CNN).

In another embodiment of the method of diagnosing a ND in a patient, the one or more MRI images includes at least one of T1 weighted structural (T1w) images. Diffusion-weighted imaging (DWI) images, magnetization transfer-weighted images, susceptibility-weighted images, Neuromelanin-sensitive MRI images, T2-weighted images, quantitative Susceptibility Mapping (QSM) images, and functional MRI (fMRI) images.

In another embodiment of the method of diagnosing a ND in a patient, the at least one training data set further includes data of ND mimics.

In another embodiment of the method of diagnosing a ND in a patient, the at least one training data set further includes data of different stages and subtypes of the ND, and wherein the method further comprises classifying the ND stage and subtype of the patient.

In another embodiment of the method of diagnosing a ND in a patient, the ND includes Parkinson's disease (PD). Alzheimer's disease (AD), amyotrophic lateral sclerosis (ALS), Multiple Systems Atrophy, Progressive Supranuclear Palsy, Corticobasal Ganglionic Degeneration Syndrome, Rapid Eye Movement Sleep Behaviour Disorder, Lewy Body Dementia, any one of the 10 sub-types of Neurodegeneration with Brain Iron Accumulation (NBIA), and Essential Tremor.

In another embodiment of the method of diagnosing a ND in a patient, the ND is Parkinson's disease (PD) and the region is at least one of the striatum, substantia nigra pars compacta/ventral tegmental area (SNc/VTA) and locus coeruleus.

In another embodiment of the method of diagnosing ND in a patient, the ND is AD and the one or more sub-cortical structures include at least the entorhinal cortex, hippocampus, the striatum (e.g., limbic sub-region), and SNc/VTA.

In another embodiment of the method of diagnosing a ND in a patient, the ND is ALS and the one or more sub-cortical structures include at least one of ventral spinal cord, primary motor cortex, brainstem, striatum, and SNc/VTA.

In another embodiment of the method of diagnosing a ND in a patient, the ND is Multiple Systems Atrophy and the one or more sub-cortical structures include at least one of the striatum, SNc/VTA, the globus pallidus, the locus coeruleus, and pons

In another embodiment of the method of diagnosing a ND in a patient, the ND is Progressive Supranuclear Palsy and the one or more sub-cortical structures include at least one of the striatum, SNc/VTA, the globus pallidus, and the midbrain.

In another embodiment of the method of diagnosing a ND in a patient, the ND is Corticobasal Ganglionic Degeneration and the one or more sub-cortical structures include at least one of the striatum, globus pallidus, locus coeruleus, and SNc/VTA.

In another embodiment of the method of diagnosing a ND in a patient, the ND is Rapid Eye Movement Sleep Behaviour Disorder and the one or more sub-cortical structures include at least one of the striatum, SNc/VTA, subthalamic nucleus, and locus coeruleus.

In another embodiment of the method of diagnosing a ND in a patient, the ND is Lewy Body Dementia and the one or more sub-cortical structures include at least one of the striatum, SNc/VTA, subthalamic nucleus, and locus coeruleus.

In another embodiment of the method of diagnosing a ND in a patient, the ND is any one of the 10 sub-types of Neurodegeneration with Brain Iron Accumulation (NBIA) and the one or more sub-cortical structures include at least one of the striatum, globus pallidus, subthalamic nucleus, and SNc/VTA.

In another embodiment of the method of diagnosing a ND in a patient, the ND is Essential Tremor and the one or more sub-cortical structures include at least one of the striatum, globus pallidus, subthalamic nucleus. SNc/VTA and the cerebellum.

In another embodiment, the present disclosure is a method of tracking rate of progression of a neurodegenerative disorder (ND) in a patient, the method comprising: (a) obtaining magnetic resonance imaging (MRI) data of the ND patient's brain, (b) using the MRI data of the ND patient's brain to segment one or more sub-cortical structures associated with the ND into sub-regions based on their structural connectivity to cortical sub-regions, (c) extracting one or more MRI features from each of the sub-regions generated by the segmentation of part (b), and (d) using one or more machine learning techniques to stage the progression of ND based on comparisons of the one or more MRI features to models developed on at least one training data set, the at least one training data set including MRI features of each of the sub-regions generated by the segmentation of ND patients whose stage of disease is known. In aspects, the cortical sub-regions are defined using a public MRI atlas. In one embodiment of the method of tracking rate of progression of a ND in a patient, the at least one training data includes prior MRI features of the ND patient. In another aspect the one or more MRI features are compared to one or more models developed using the at least one training data set. In another aspect, the one or more features are compared to (a) one or more models developed using the at least one training data set and (b) the at least one training data set. In another aspect the one or more MRI images of the patient's brain is one or more MRI images of the patient's cortex.

In another embodiment of the method of tracking rate of progression of a ND in a patient, the one or more MRI features include measures of surface area, surface displacement [relative to average shape of age-matched HC group], volume, connectivity, and quantitative MRI parameters, the latter enabling estimation of heavy metals, such as iron, BOLD signal, and those features identified through CNN of sub-cortical sub-regions.

In another embodiment of the method of tracking rate of progression of a ND in a patient, the MRI data includes at least one of T1 weighted structural (T1w) images, Diffusion-weighted imaging (DWI) images, magnetization transfer-weighted images, susceptibility-weighted images, T2-weighted images, quantitative Susceptibility Mapping (QSM) images, Neuromelanin-sensitive MRI images and fMRI images.

In another embodiment of the method of tracking rate of progression of a ND in a patient, the at least one training data set further includes data of ND mimics.

In another embodiment of the method of tracking rate of progression of a ND in a patient, the at least one training data set further includes data of different stages and subtypes of the ND, and wherein the method further comprises classifying the ND stage and subtype of the patient.

In another embodiment, the present disclosure is a method of prognosticating the symptoms and severity of a neurodegenerative disorder (ND), that will develop in a patient, the method comprising: (a) obtaining MRI data of the patient's brain (including the patient's cortex), such as T1 weighted structural MRI (T1w) data, Diffusion-weighted imaging (DWI) data, magnetization transfer weighted images, Quantitative Susceptibility Mapping, Neuromelanin-sensitive MRI images of the ND patient's brain, and/or fMRI (b) using the MRI data of the patient's brain (including the cortex) to segment one or more sub-cortical structures associated with the ND into sub-regions based on their structural connectivity to cortical sub-regions, allowing focused measurements of those cortical sub-regions within the above-mentioned sub-cortical structures that are most predictive of/associated with symptoms of the ND that produce more malignant disease, (c) extracting one or more MRI features from each of the sub-regions, and (d) using one or more machine learning techniques to subtype ND based on comparisons of the one or more MRI features to at least one training data set, the at least one training data sets including MRI features of each of the sub-regions generated by the segmentation of ND patients whose symptoms of disease are known. In another aspect, the one or more MRI features are compared to one or more models developed using the at least one training data set. In another aspect, the one or more features are compared to (a) one or more models developed using the at least one training data set and (b) the at least one training data set.

In one embodiment of the method of tracking rate of progression of a ND or prognosticating the symptoms and severity of a ND, the ND includes Parkinson's disease (PD), Alzheimer's disease (AD) and amyotrophic lateral sclerosis (ALS), Multiple Systems Atrophy, Progressive Supranuclear Palsy, Corticobasal Ganglionic Degeneration, Rapid Eye Movement Sleep Behaviour Disorder, Lewy Body Dementia, any one of the 10 sub-types of Neurodegeneration with Brain Iron Accumulation (NBIA) and Essential Tremor.

In another embodiment of the method of tracking rate of progression of a ND or prognosticating of the symptoms and severity of a ND, the ND is Parkinson's disease (PD) and the one or more sub-cortical structures include at least one of the striatum, SNc/VTA, and locus coeruleus.

In another embodiment of the method of tracking rate of progression of a ND or prognosticating the symptoms and severity of a ND, the ND is AD and the one or more sub-cortical structures include at least the entorhinal cortex, hippocampus, the striatum (e.g., limbic sub-region), and SNc/VTA.

In another embodiment of the method of tracking rate of progression of a ND or prognosticating the symptoms and severity of a ND, the ND is ALS and the one or more sub-cortical structures include at least one of ventral spinal cord, primary motor cortex, brainstem, striatum, and SNc/VTA.

In another embodiment of the method of tracking rate of progression of a ND or prognosticating the symptoms and severity of a ND, the ND is Multiple Systems Atrophy and the one or more sub-cortical structures include at least one of the striatum, SNc/VTA, the globus pallidus, the locus coeruleus, and pons

In another embodiment of the method of tracking rate of progression of a ND or prognosticating the symptoms and severity of a ND, the ND is Progressive Supranuclear Palsy and the one or more sub-cortical structures include at least one of the striatum, the globus pallidus, the midbrain, and SNc/VTA.

In another embodiment of the method of tracking rate of progression of a ND or prognosticating the symptoms and severity of a ND, the ND is Corticobasal Ganglionic Degeneration and the one or more sub-cortical structures include at least one of the striatum, globus pallidus, locus coeruleus, and SNc/VTA.

In another embodiment of the method of tracking rate of progression of a ND or prognosticating the symptoms and severity of a ND, the ND is Rapid Eye Movement Sleep Behaviour Disorder and the one or more sub-cortical structure include at least one of the striatum, subthalamic nucleus, locus coeruleus, and SNc/VTA.

In another embodiment of the method of tracking rate of progression of a ND or prognosticating the symptoms and severity of a ND, the ND is Lewy Body Dementia and the one or more sub-cortical structures include at least one of the striatum, subthalamic nucleus, locus coeruleus, and SNc/VTA.

In another embodiment of the method of tracking rate of progression of a ND or prognosticating the symptoms and severity of a ND, the ND is any one of the 10 sub-types of Neurodegeneration with Brain Iron Accumulation (NBIA) and the one or more sub-cortical structures include at least one of the striatum, globus pallidus, subthalamic nucleus, and SNc/VTA.

In another embodiment of the method of tracking rate of progression of a ND or prognosticating the symptoms and severity of a ND, the ND is Essential Tremor and the one or more sub-cortical structures include at least one of the striatum, globus pallidus, subthalamic nucleus, SNc/VTA, and the cerebellum.

In one embodiment of any of the methods of this disclosure, the method is cloud based or computer based.

In another embodiment, the present disclosure provides for a system to diagnose a neurodegenerative disorder (ND) in a subject, the system, in one embodiment, comprising: (a) a database comprising control ND MRI image features based on ND image diagnosis, and/or control non-ND MRI image features based on non-ND image diagnosis, (b) a processor configured to receive the database and MRI images of the subject's brain, (c) one or more machine learning techniques operatively coupled to the processor, the one or more machine learning techniques being trained with the database to obtain one or more trained machine learning techniques, and (d) a computer program product connected to the processor, the computer program product comprising a non-transitory computer readable storage medium and a computer program mechanism embedded therein, the computer program mechanism comprising executable instructions for diagnosing the ND in the subject, the instructions, when executed by the processor, cause the processor to perform the following operations: (i) using the MRI images of the subject's brain to segment one or more sub-cortical structures associated with the ND into sub-regions based on structural connectivity of the sub-regions to cortical sub-regions, (ii) extracting one or more MRI features from each of the sub-regions generated by the segmentation, and (iii) testing the trained one or more machine learning techniques with the extracted one or more MRI features to classify the patient as being ND positive or ND negative. In one aspect, the database further includes MRI features of each of the sub-regions generated by the segmentation of ND patients whose stage and symptoms of disease are known, and wherein the operations further include estimating stage of the progression of the ND and prognosticating symptoms and severity of the ND that will develop in the patient. In aspects, the cortical sub-regions are defined using a public MRI atlas. In another aspect, the one or more machine learning techniques are trained with one or more models developed using the database. In another aspect, the one or more machine learning techniques are trained with (a) the database and (b) the one or more models developed using the training dataset.

In another embodiment, the present disclosure relates to a cloud-based system to diagnose a neurodegenerative disorder (ND) in a subject, comprising: (a) a database stored in the cloud, the database comprising control ND MRI image features based on ND image diagnosis, and/or control non-ND MRI image features based on non-ND image diagnosis, and (b) an analysis pipeline configured for: (i) training one or more machine learning techniques with the database to obtain one or more trained machine learning technique, (ii) using MRI images of the subject's brain to segment sub-cortical structures associated with the ND into sub-regions based on structural connectivity of the sub-regions to cortical regions, (iii) extracting one or more MRI features from each of the sub-regions generated by the segmentation, and (vi) testing the one or more trained machine learning technique with the extracted one or more MRI features to classify the subject as being ND positive or ND negative. In one aspect, the database further includes MRI features of each of the sub-regions generated by the segmentation of ND patients whose stage and symptoms of disease are known, and wherein the analysis pipeline is configured to estimate stage of the progression of the ND and for prognosticating symptoms and severity of the ND that will develop in the patient. In aspects, the cortical sub-regions are defined using a public MRI atlas. In another aspect the one or more machine learning techniques are trained with one or more models developed using the database. In another aspect, the one or more machine learning techniques are trained with (a) the database and (b) the one or more models developed using the training dataset.

In one embodiment of the systems of the present disclosure, the ND includes Parkinson's disease (PD), Alzheimer's disease (AD) and amyotrophic lateral sclerosis (ALS), Multiple Systems Atrophy, Progressive Supranuclear Palsy, Corticobasal Ganglionic Degeneration, Rapid Eye Movement Sleep Behaviour Disorder, Lewy Body Dementia, any one of the 10 sub-types of Neurodegeneration with Brain Iron Accumulation (NBIA) and Essential Tremor.

### BRIEF DESCRIPTION OF THE DRAWINGS

The following figures illustrate various aspects and preferred and alternative embodiments of this disclosure.

FIG. 1. Sub-cortical structures in the brain.

FIG. 2. Schematic of connectivity-driven subject-specific parcellation of striatal and SNc/VTAsub-regions and extracted features. The CIT168 probabilistic sub-cortical atlas defined the striatal, SNc/VTA ROIs, which were parcellated into sub-regions according to their tractography-based connection profiles to cortical sub-regions. These striatal sub-regions are implicated in different functions, and each are affected distinctly by PD, which are important motives for considering them separately.

Six features extracted from these sub-regions are listed onto the right: 1) volumes of striatal sub-regions, 2) striatum to target connectivity, 3) FA along pathways, 4) MD along pathways, 5) surface areas of each striatal sub-regions, 6) surface displacement: inward (cool colours) and outward (warm colours) relative to an average template based on healthy elderly controls.

FIG. 3. AUC curves from eight independent experiments classifying PD and healthy controls using XGBoost.

FIG. 4. AUC from the best performing model among eight experiments classifying PD and healthy controls using XGBoost.

FIG. 5. AUC curves from three independent experiments classifying early-stage PD (<12 month duration of illness) and healthy controls using neural network analysis.

FIG. 6. AUC curves from three independent experiments classifying early-stage PD (<24 month duration of illness) and healthy controls using neural network analysis.

FIGS. 7A and 7B. AUC curves classifying RBD patients and healthy controls (7A) and PD and healthy controls (7B) using machine learning and iron studies.

### DETAILED DESCRIPTION OF EXEMPLARY EMBODIMENTS

### Abbreviations

Unless defined otherwise, all technical and scientific terms used herein have the same meaning as commonly understood by one of ordinary skill in the art to which this disclosure belongs. Also, unless indicated otherwise, except within the claims, the use of “or” includes “and” and vice versa. Non-limiting terms are not to be construed as limiting unless expressly stated or the context clearly indicates otherwise (for example “including”, “having” and “comprising” typically indicate “including without limitation”). Singular forms including in the claims such as “a”, “an” and “the” include the plural reference unless expressly stated otherwise. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this disclosure.

All numerical designations, e.g., levels, amounts and concentrations, including ranges, are approximations that typically may be varied (+) or (−) by increments of 0.1, 1.0, or 10.0, as appropriate. All numerical designations may be understood as preceded by the term “about”.

“Baseline” means a measurement of reference in a subject. In embodiments, the term may include measurements, such as MRI measurements, taken within about six months of their PD diagnosis by standard diagnostic procedures and prior to initiation of dopaminergic therapy. In other embodiments, the term may include a measurement taken before the subject is diagnosed as having PD by standard diagnostic procedures.

Magnetic resonance imaging (MRI) is based on imaging water rich soft central nervous tissue. The MRI data acquisition involves water spin polarization or alignment in a strong magnetic field and then the application of timed and controlled spatially dependent magnetic pulses for spatial encoding. The signal is collected using a radio-frequency tuned near-field coil and then amplified, decoded and visualized to show the water density maps. The MRI contrast can be used to differentiate different tissue types (e.g., gray matter, myelinated white matter and cerebrospinal fluid or abnormal tissue (e.g., demyelination, tumors, and infarcts).

Diffusion tensor imaging (DTI) or diffusion tensor magnetic resonance imaging (DTMRI) uses the same MRI data acquisition and processing. In addition to the standard MRI acquisition paradigm, strong diffusion magnetic pulses (Gx, Gy, Gz) or (gx, gy, gz) are applied along the three gradient channels to obtain diffusion-weighted or contrasted data.

The term “patient” as used herein refers to a subject that is suspected of having Parkinson's Disease (PD).

The term “subject” as used herein refers all members of the animal kingdom including mammals, preferably humans.

The controls used in the present disclosure, including ND positive patients, ND negative subjects, ND stages, ND symptoms and so forth, are known through clinical information and standard clinical measurements by medical specialists.

### Overview

Embodiments of the disclosure as described herein generally include methods for performing training and recognition of neurodegenerative disorder (ND) in a subject. The present disclosure provides for objective diagnostic, progression, or prognostic tests to identify patients with ND on an individual basis (i.e., to accurately recognize or classify patients and controls at the single-subject level). Accordingly, while the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the disclosure.

The present disclosure uses a) structural, diffusion, iron/neuromelanin, and/or functional MRI, b) automated imaging analysis, segmentation, and diagnostic/progression/prognosis pipeline with tractography that i) segments sub-cortical structures that lack visible internal boundaries on MRI using tractography, ii) extracts sub-cortical sub-regional measures, and iii) applies one or more machine learning technique to combine extracted measures and, in aspects of the disclosure, to establish models that discriminate between patients with different NDs, and/or healthy controls, as well as to track and predict disease sub-type, progression, and severity. In embodiments, the diagnostic, progression, and prognostic biomarkers arising from machine learning methods in this disclosure are automated. They can be applied to individual MRI scans by clinicians or other researchers who lack any expertise in image analysis, modelling, or machine learning techniques to obtain information about diagnosis, staging, sub-typing, or predicted evolution of ND(s).

In one embodiment, the present disclosure is a method of diagnosing a neurodegenerative disorder (ND) in a subject or patient, the method comprising: (a) obtaining one or more magnetic resonance imaging (MRI) images of the patient's brain, (b) implementing a processing pipeline configured to (i) use data from the one or more MRI images of the patient's brain to segment sub-cortical structures associated with the ND into sub-regions based on their structural connectivity to cortical regions, allowing focused measurements of those sub-regions within the sub-cortical structures that are most affected by change in motor and non-motor symptoms, (ii) extracting one or more MRI features from each of the sub-cortical regions generated by the segmentation of (b) (i) of the patient's brain, (iii) feeding the one or more MRI features to one or more machine learning techniques, and (iv) using the one or more machine learning techniques to classify the patient as being ND positive or ND negative based on comparisons of the one or more MRI features of the subject's/patient's brain to at least one training data set, the at least one training data set including data of known ND positive controls and ND negative controls, thereby diagnosing ND in the patient. The ND positive controls and ND negative controls may be obtained for example through clinical information and standard clinical measurements by medical specialists. In aspects, the cortical sub-regions are defined using a public MRI atlas. In another aspect the one or more MRI features are compared to one or more models developed using the at least one training data set. In another aspect, the one or more features are compared to (a) one or more models developed using the at least one training data set and (b) the at least one training data set. In another aspect the one or more MRI images of the patient's brain is one or more MRI images of the patient's cortex.

In one embodiment, the method is implemented by a computer or cloud based.

In one embodiment, the MRI image of any of the embodiments disclosed in this disclosure includes at least one of T1 weighted structural (T1w) images, Diffusion-weighted imaging (DWI) images, magnetization transfer-weighted images (neuromelanin-sensitive MRI), susceptibility-weighted images, T2-weighted images, and quantitative Susceptibility Mapping (QSM) images, Neuromelanin-sensitive MRI images of the subject's/patient's brain. In embodiments, quantitative model-based estimates of intrinsic tissue parameters using weighted images are used, e.g., DWI to evaluate neurite density index, or T1w, T2w to study myelin mapping index. In embodiments, measures of iron are obtained (QSM/R2*).

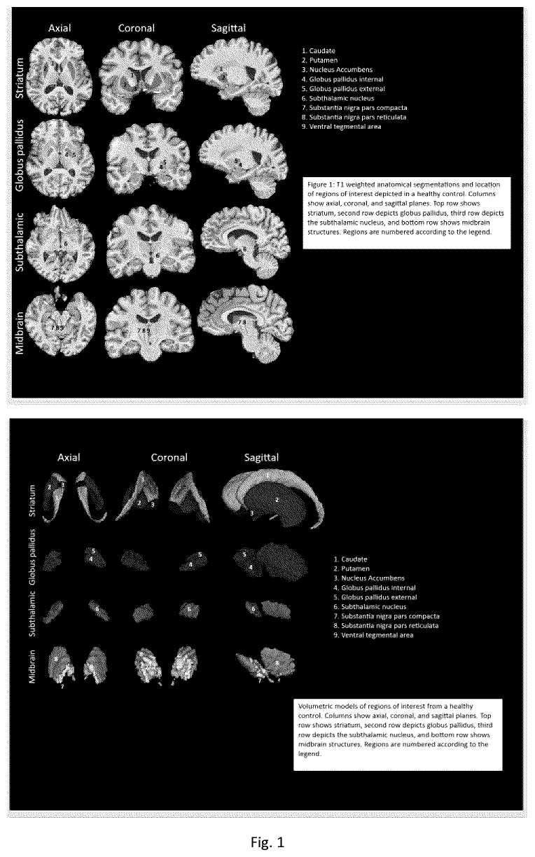

A processing pipeline processes data from the MRI image taken from the subject/patient to segment sub-cortical regions associated with the ND based on their structural connectivity to cortical sub-regions, which are distinguished by their functions. In embodiments, to ensure that every step of the segmentation process is objectively specified, entirely reproducible, and not dependent upon expertise of users (e.g., accurate detection and manual tracing of structures), the cortex is partitioned into seven regions using explicit coordinates from a publicly available MRI atlas, such as Harvard-Oxford neuroimaging atlas. Next, processing pipeline segments the region associated with the ND, such as the striatum, substantial nigra, ventral tegmental area and the locus coruleus in the case of PD, into sub-regions dictated by the cortical regions to which they were predominantly connected, using, for example, probabilistic tractography—a method for tracing white matter tracks (see Connectivity-based parcellation of the striatum: Volume and connectivity measures, for further details). In the case of the striatum, this segmentation enabled targeted measures of striatal sub-regions that are predicted to be most sensitive to PD (see FIG. 1).

From each segmented sub-cortical region, at least one measure related to the one or more MRI features, such as surface area, surface displacement [relative to average shape of age-matched HC group], volume, connectivity, and quantitative MRI parameters, the latter enabling estimation of heavy metals such as iron, are extracted as separate features.

Machine learning techniques are used to determine the best combination of extracted features for diagnosing the ND using structural MRI. In machine learning, preconceived expectations are averted and through a process of optimization, a model emerges, is fitted, and fine-tuned, connecting inputs—MRI features in this case—and outputs—ND versus control categorization here.

Examples of suitable machine learning techniques or models include, for example, Supervised Learning, Unsupervised Learning, and Reinforcement Learning. Examples of machine learning algorithms include linear regression, decision trees, random forest (RF) classifiers, and XGBoost.

The machine learning technique is trained with a training data set such as one or more MRI features of known ND positive controls and with MRI features of ND negative controls. A diagnosis of the subject/patient is obtained by testing the trained machine learning technique with the one or more MRI features of the subject. In aspects, the training data set includes control data of ND mimics, thereby distinguishing the ND from ND mimics. The controls may be obtained for example through clinical information and standard clinical measurements by medical specialists.

The reproducibility and generalizability of the methods of the present disclosure has been demonstrated in an entirely independent sample of medicated PD patients (mean disease duration <2.5 years) and healthy controls. Classification accuracy was 95%. As such, the present disclosure provides a first MRI diagnostic of PD that is highly accurate and consistent, relying on an automated analysis pipeline that can be readily translated to PD diagnosis, research and practice.

In embodiments, the training data set further includes control data of different stages and subtypes of the ND, thus allowing classifying the ND stage and subtype of the patient.

ND includes Parkinson's disease (PD), Alzheimer's disease (AD) and amyotrophic lateral sclerosis (ALS), Multiple Systems Atrophy, Progressive Supranuclear Palsy, Corticobasal Ganglionic Degeneration, Rapid Eye Movement Sleep Behaviour Disorder, Multiple Systems Atrophy. Progressive Supranuclear Palsy, Corticobasal Ganglionic Degeneration, Rapid Eye Movement Sleep Behaviour Disorder, Lewy Body Dementia, any one of the 10 sub-types of Neurodegeneration with Brain Iron Accumulation (NBIA) and/or Essential Tremor.

In embodiments, the ND is Parkinson's disease (PD) and the sub-cortical structure is at least one of the striatum, substantia nigra, ventral tegmental area and locus coeruleus.

In embodiments, the ND is AD and the sub-cortical structure includes at least the entorhinal cortex and hippocampus.

In embodiments, the ND is ALS, and the sub-cortical structure includes at least one of ventral spinal cord, primary motor cortex and brainstem.

In embodiments, the ND is Multiple Systems Atrophy and the sub-cortical structure includes at least one of the striatum, the globus pallidus, the locus coeruleus, and pons.

In embodiments, the ND is Progressive Supranuclear Palsy and the sub-cortical structure includes at least one of the striatum, the globus pallidus, the midbrain, substantia nigra, and ventral tegmental area

In embodiments, the ND is Corticobasal Ganglionic Degeneration and the sub-cortical structure includes at least one of the striatum, globus pallidus, locus coeruleus, substantia nigra, and ventral tegmental area.

In embodiments, the ND is Rapid Eye Movement Sleep Behaviour Disorder and the region includes at least one of the striatum, subthalamic nucleus, locus coeruleus, substantia nigra, and ventral tegmental area.

In embodiments, the ND is Lewy Body Dementia and the sub-cortical structure includes at least one of the striatum, subthalamic nucleus, locus coeruleus, substantia nigra, and ventral tegmental area.

In embodiments, the ND is any one of the 10 sub-types of Neurodegeneration with Brain Iron Accumulation (NBIA) and the sub-cortical structure includes at least one of the striatum, globus pallidus, subthalamic nucleus, substantia nigra, and ventral tegmental area.

In embodiments, the ND is Essential Tremor and the sub-cortical structure includes at least one of the striatum, globus pallidus, subthalamic nucleus, substantia nigra, ventral tegmental area, and the cerebellum.

In embodiments, the present disclosure provides method, including computer-implemented or cloud implemented methods, for tracking the rate of progression of a neurodegenerative disorder (ND) in a subject or ND patient, the method comprising: (a) obtaining a magnetic resonance imaging (MRI) data of the ND patient's brain, (b) using the MRI data of the ND patient's brain to segment sub-cortical structures associated with the ND into sub-regions based on their structural connectivity to cortical regions, which are distinguished by their functions, (c) extracting one or more MRI features from each of the sub-cortical regions generated by the segmentation of part (b), and (d) using the machine learning to stage the progression of PD based on comparisons of the one or more MRI features to a training data set, the training data set including MRI data of the one or more sub-regions of ND patients whose stage of disease is known (for example through clinical information and standard clinical measurements by medical specialists).

In embodiments, the present disclosure provides for a method of prognosticating the symptoms and severity of a neurodegenerative disorder (ND), such as Parkinson's disease (PD), that will develop in a patient, the method comprising: (a) receiving MRI data, such as T1 weighted structural MRI (T1w) data, DWI MRI (DWI) data, magnetization transfer-weighted images, Neuromelanin-sensitive MRI images, and/or Quantitative Susceptibility Mapping of the ND patient's brain, (b) using the MRI data of the patient's brain to segment sub-cortical structures associated with the ND into sub-regions based on their structural connectivity to cortical regions, which are distinguished by their functions, allowing focused measurements of those sub-regions within the above-mentioned sub-cortical structures that are most predictive of/associated with symptoms of the ND that produce with more malignant disease. (c) extracting one or more MRI features from each of the sub-regions, and (d) using a machine learning technique to subtype ND based on comparisons of the one or more MRI features to a training data set, the training data set including MRI data of the one or more sub-regions of ND patients whose symptoms of disease are known (for example through clinical information and standard clinical measurements by medical specialists).

Upon positive ND diagnosis of a subject by the methods of the present invention, the subject is treated for said ND. The treatment is tailored to the particular ND, subtype of ND and progression of ND. In PD, dopaminergic therapies such as levodopa, dopamine precursor, or pramipexole, a dopamine agonist, will alleviate symptoms, though no therapies, at present, change the course of the disease.

In another embodiment, the present disclosure provides for a system to diagnose a neurodegenerative disorder in a subject. In one embodiment, the system comprises: (a) a database comprising control ND MRI image features based on ND image diagnosis, and control non-ND MRI image features based on non-ND image diagnosis, (b) a processor configured to receive the database and MRI images of the subject's brain, (c) a machine learning technique operatively coupled to the processor, the machine learning technique being trained with the database, and (d) a computer program product connected to the processor, the computer program product comprising a non-transitory computer readable storage medium and a computer program mechanism embedded therein, the computer program mechanism comprising executable instructions for diagnosing the ND in the subject, the instructions, when executed by the processor, cause the processor to perform the following operations: (i) using the MRI images of the subject's brain to segment sub-cortical structures associated with the ND into sub-regions based on structural connectivity of the sub-regions to cortical regions, which are distinguished by their functions, (ii) extracting one or more MRI features from each of the sub-regions generated by the segmentation, and (iii) testing the trained machine learning technique with the extracted one or more MRI features to classify the patient as being ND positive or ND negative.

In another embodiment, the system to diagnose a neurodegenerative disorder in a subject is cloud-based and comprises: (a) a database stored in the cloud, the database comprising control ND MRI image features based on ND image diagnosis, and control non-ND MRI image features based on non-ND image diagnosis, and (b) an analysis pipeline configured for: (i) training a machine learning technique with the database to obtain a trained machine learning technique, (ii) using MRI images of the subject's brain to segment sub-cortical structures associated with the ND into sub-regions based on structural connectivity of the sub-regions to cortical regions, which are distinguished by their functions, (iii) extracting one or more MRI features from each of the sub-regions generated by the segmentation, and (vi) testing the trained machine learning technique with the extracted one or more MRI features to classify the subject as being ND positive or ND negative.

In embodiments of the system to diagnose a neurodegenerative disorder in a subject, the system further comprises one or more MRI atlases, and the cortical sub-regions are defined using said one or more MRI atlases.

In embodiments of the system to diagnose a neurodegenerative disorder in a subject, all aspects and analyses done by the system are automated, including training, imaging analysis, extracting, segmentation, testing and diagnostic/progression/prognosis, and including defining the cortical sub-regions by comparison to the one or more MRI atlases.

In embodiments of the system to diagnose a neurodegenerative disorder in a subject, one or more aspects and analyses done by the system are automated, including training, imaging analysis, extracting, segmentation, testing and diagnostic/progression/prognosis, and including defining the cortical sub-regions by comparison to the one or more MRI atlases.

In embodiments of the cloud-based system, the system further comprises one or more MRI atlases stored in the cloud, and the cortical sub-regions are defined said one or more public atlases.

In embodiments of the cloud-based system, all aspects and analyses done by the system are automated, including training, imaging analysis, extracting, segmentation, testing and diagnostic/progression/prognosis, and including defining the cortical sub-regions by comparison to the one or more MRI atlases stored in the cloud.

In embodiments of the cloud-based system, one or more aspects and analyses done by the system are automated, including training, imaging analysis, extracting, segmentation, testing and diagnostic/progression/prognosis, and including defining the cortical sub-regions by comparison to the one or more MRI atlases stored in the cloud.

In embodiments of the system to diagnose a neurodegenerative disorder in a subject and of the could-based system, the database further includes MRI features of each of the sub-regions generated by the segmentation of ND patients whose stage and symptoms of disease is known, and wherein the operations further include, or the analysis pipeline is further configured, to estimate stage of the progression of the ND and to prognosticate regarding symptoms that will emerge and severity of the ND that will develop in the patient.

The database referred to in the above embodiments comprises libraries of predetermined MRI features from the sub-regions of ND positive controls and ND negative controls, of patients whose stage and symptoms of ND is known or verified, may be provided in a computer product (memory sticks, as an app for handheld devices such as pads and cellular phones or accessible in a cloud-based application and so forth), or they may be uploaded to the memory of a computer system, including main frames, desktops, laptops, hand-held devices, such as pads, smart watches, and cellular phones, or they may be stored in the cloud. MRI features of the one or more sub-regions as explained above may be taken from a subject suspected of having a ND. The subject's MRI features may then be uploaded to the computer system (main frames, desktops, lab tops, handheld devices (pads, smart telephones, smart watches and so forth), or a cloud-based application. An operator may then compare the subject's MRI features with the predetermined MRI features of ND (ND positive controls) and non-ND (normal controls) using machine learning to determine not only if the subject has ND, but also the subtype of ND and predicted severity of ND. The operator may select the type/model of machine learning that is used.

In the case of PD, in clinical practice, because therapy is only symptomatic, it does not change the progression of the disease. In this way, the evolution of structural changes that indicate PD progression will not be altered whether or not symptomatic treatment has been started. A key advantage of measuring the progression of PD in accordance with the present disclosure is that measurement of progression is important for clinical researchers who are trying to identify treatments that are disease-modifying (i.e., that could alter the progression of PD).

The ND for any of the above-mentioned embodiments includes Parkinson's disease (PD), Alzheimer's disease (AD) and amyotrophic lateral sclerosis (ALS), Multiple Systems Atrophy, Progressive Supranuclear Palsy, Corticobasal Ganglionic Degeneration and/or Rapid Eye Movement Sleep Behaviour Disorder. The sub-cortical structures have been described above for each ND.

In the case of PD the sub-cortical structures are associated with more malignant disease such as akinetic-rigid sub-type, freezing of gait, cognitive impairment, severe anxiety, severe depression, rapid eye movement sleep behaviour disorder, orthostatic hypotension, dyskinesias, hallucinations.

In order to aid in the understanding and preparation of the within disclosure, the following illustrative, non-limiting, examples are provided.

### Examples

### Materials and Methods

### 1.0 Participants

### 1.1 Recruitment

University of Western Ontario (i.e., Western/UWO): Participants with Parkinson's Disease (PD) were recruited through the Movement Disorder Database at the London Health Sciences Centre at the University of Western Ontario and healthy control volunteers were recruited through the local community. All patients with PD had their diagnosis confirmed by a movement disorder neurologist using MDS criteria (or previously published criteria [ie. UK Brain Bank] if patients were diagnosed before the publication of the MDS criteria). Twenty-one patients with rapid eye movement (REM) behaviour sleep disorder (RBD), a prodromal Parkinsonian disorder, were also recruited. RBD patients were diagnosed by physicians at the Sleep Disorders Clinic at the London Health Sciences Centre based on video polysomnography and the appropriate diagnostic criteria.

Montreal Neurological Institute/Hospital (i.e., MNI)/Quebec Parkinson's Network (QPN): Participants with PD were recruited from the MNI. Though the Centre Hospitalier de I'Université de Montréal (CHUM), the Centre Hospitalier Universitaire de Québec (CHUQ), and the CHU de Québec-Université Laval Research Centre are also participating centres in the QPN, only PD patients and healthy controls from the MNI were included in the current study. All patients were diagnosed by a movement disorders specialist using MDS criteria (or previously published criteria [ie. UK Brain Bank] if patients were diagnosed before the publication of the MDS criteria), with an average Hoehn and Yahr stage of 2.35.

Ontario Neurodegenerative Disease Research Initiative (ONDRI): Participants with PD were recruited from multiple sites across Ontario including London Health Sciences Centre, Sunnybrook Health Sciences Centre, St. Michael's Hospital, The Ottawa Hospital, and Toronto Western Hospital. Patients with PD were diagnosed based on UK Brain Bank criteria. Patients with mild cognitive impairment and amyotrophic lateral sclerosis were also recruited through this initiative and included in this study.

Calgary: Participants with PD were recruited from the University of Calgary's Movement Disorders program, and healthy control volunteers were recruited from the community. Patients with PD were diagnosed according to the UK brain bank criteria for idiopathic PD.

### 2.0 Data Acquisition

### 2.1 Demographic, Clinical, and Behavioural Data

Western: Participants completed self-report questionnaire measures of anxiety, depression, apathy, impulsivity, freezing of gait, happiness, and sleepiness. Participants also completed the Montreal Cognitive Assessment and Unified Parkinson's Disease Rating Scale-III (UPDRS) motor assessment.

MNI/QPN: Participants completed the QPN Questionnaire with assistance from a neurologist or a trained research assistant. The information gathered from this questionnaire includes demographic and clinical information (e.g., diagnosis, motor symptoms, treatment and medications, family history of PD). Participants also completed cognitive tests, magnetoencephalography (MEG), neuropsychological evaluations, motor evaluations, and provided a recorded speech sample.

ONDRI: Participants completed neuropsychological, gait, and ocular assessments; and provided a blood sample for neurodegeneration-related genomic analysis. Additionally, patients provide baseline family history and detailed demographic information, and undergo annual clinical assessments that include neurological examination, the Montreal Cognitive Assessment, vital signs, neuropsychiatric evaluation, UPDRS, as well as sleep, quality of life, and disability impact questionnaires.

Calgary: Participants completed a battery of neuropsychological tests across five domains (executive function, attention, language, visuo-spatial, memory), in addition to the Montreal Cognitive Assessment. Motor symptom severity for patients with PD was evaluated using the Unified Parkinson's Disease Rating Scale-III (UPDRS). Participants also completed cognitive tasks while undergoing fMRI (e.g., face associated scene task, Wisconsin card sorting task).

### 2.2 Image Acquisition

Western: Participants were scanned on a 3T Siemens MAGNETOM Prisma Fit whole-body scanner at the Centre for Functional and Metabolic Mapping, Western University, London, Ontario, Canada. The scanner had a 32-receiver channel head coil with head position fixation devices installed and a standard body transmit coil was used. A localizer image was obtained first to position participants. T1-weighted (T1w) anatomical scans were obtained for structural information, registration of dMRI scans, and the segmentation of VTA/SNc and striatum using the CIT168 probabilistic subcortical atlas. T1w anatomical images were acquired using a magnetization-prepared rapid gradient echo (MPRAGE) sequence [repetition time (TR)=2300 ms, echo time (TE)=2.98 ms, flip angle=9°, Field of View (FoV)=256×256 mm2, 159 slices, voxel size=1×1×0.9 mm3, receiver bandwidth=160 Hz/Px, acquisition time=5:35 min]. dMRI scans were acquired for parcellation through probabilistic tractography and generation of imaging features for group level comparisons. All dMRI scans were acquired using an echo-planar imaging sequence (TR=3800 ms, TE=88 ms, flip angle=90°, gradient directions=95, b1-value of 1000 s/m2, b2-value of 2000 s/mm2, FoV=232×232 mm2, 72 slices, voxel size=2×2×2 mm3, receiver bandwidth=1488 Hz/Px, acquisition time=7:02 min). Two sequences with reversed phase encoding direction were acquired to correct for susceptibility induced distortions.

For some PD patients, RBD patients, and healthy controls, in whom iron estimation was sought, high resolution gradient echo (GRE) images were acquired with an rf-spoiled, flow compensated 3D gradient echo sequence with six echoes (TE 8.09 ms to 40.49 ms with an interval of 6.48 ms), and (TR=52 ms, flip angle=20°. FoV=224×224 mm², 96 slices, voxel size=0.5×0.5×2 mm³, receiver bandwidth=160 Hz/Px, acquisition time=8:30 min) to generate QSM images (Jenkinson et al., 2012).

MNI/QPN: Patients at the MNI underwent T1-weighted imaging with a 3T Siemens TIM Trio scanner with a 12-channel head coil, MPRAGE sequence: repetition time (TR): 2300 ms, echo time (TE): 2.91 ms, flip angle: 9° and voxel size: 1 mm³ isotropic. The Paris cohort underwent T₁-weighted imaging with a 3 T Siemens TIM Trio scanner with a 12-channel head coil, MPRAGE sequence: TR: 2300 ms, TE: 4.18 ms, inversion time (TI): 900 ms, flip angle: 9° and voxel size: 1 mm³ isotropic; or a 3 T PRISMA Fit scanner with a 64-channel head coil, MP2RAGE sequence: TR: 5000 ms, TE: 2.98 ms, TI: 700 and 2500 ms, flip angle: 4° and 5°, GRAPPA: 3 and voxel size: 1 mm³ isotropic.

ONDRI: MR images of PD patients were acquired on Siemens and General Electric (GE) scanners at sites located throughout Ontario. Quality control and imaging were performed in accordance with the ONDRI guidelines.

Patients at London Health Sciences Centre (Western) were scanned on a 3T Siemens MAGNETOM Prisma Fit whole-body scanner at the Centre for Functional and Metabolic Mapping, Western University, London, Ontario, Canada. The scanner had a 32-receiver channel head coil with head position fixation devices installed and a standard body transmit coil was used. A localizer image was obtained first to position participants. Three-dimensional Ti-weighted anatomical scan (1 mm isotropic resolution) was used for volumetric assessment of brain structures, proton density (PD)/T₂-weighted scan (resolution time [TR]=3000, echo time 1 [TE₁]˜10 ms, TE₂˜90-100 ms, 3 mm thick interleaved) used for the assessment of tissue ischemia and skull stripping, fluid-attenuated inversion recovery (TR=9000 ms, TI˜2250-2500 ms) for the assessment of white matter hyperintensities, gradient echo (TR=650 ms, TE=20 ms) for the assessment of tissue microbleeds, resting state functional MRI (TR=2400 ms, TE=30 ms, flip angle=70°, 3.5 mm isotropic resolution, 250 volumes, 10-minute acquisition time) for the evaluation of brain network activity, and finally diffusion tensor imaging (2 mm isotropic resolution, 30 to 32 directions, b-value=1000) for the evaluation of white matter structural integrity.

Participants at Sunnybrook Health Sciences Centre were scanned on a GE 3.0 Tesla Discovery MR750. T1 imaging was acquired using a 3D Fast Low Angle Shot SPoiled Gradient-Recalled (3D FAST SPGR) sequence (repetition time (TR)=8.156 ms, echo time (TE)=3.18 ms, flip angle=11°, Field of View (FoV)=256×256 mm2, 176 slices, voxel size=1×1×1 mm3, pixelBandwidth=244.141 Hz/Px, acquisition time=5:22 min). dMRI scans were acquired using an echo-planar imaging sequence (TR=9000 ms. TE=82:89 ms, flip angle=90°, gradient directions=30, b-value 1=0, b-value 2=1000, FoV=128×128 mm2, 2310 slices, voxel size=2×2×2 mm3, acquisition time=6:30 min).

Participants at Toronto Western Hospital were scanned on a GE 3.0 Tesla Sigma HDxt. T1 imaging was acquired using a 3D Fast Low Angle Shot SPoiled Gradient-Recalled (3D FAST SPGR) sequence (repetition time (TR)=6.9 ms, echo time (TE)=2.8 ms, flip angle=11°, Field of View (FoV)=256×256 mm2, 176 slices, voxel size=1×1×1 mm3, pixelBandwidth=244.141 Hz/Px, acquisition time=5:22 min). dMRI scans were acquired using an echo-planar imaging sequence (TR=11700 ms, TE=105:110 ms, flip angle=90°, gradient directions=30, b-value 1=0, b-value 2=1000, FoV=128×128 mm2, 2310 slices, voxel size=2×2×2 mm3, acquisition time=6:30 min).

Participants at St. Michael's Hospital were scanned on a Siemens 3.0 Tesla Skyra. T1 imaging was acquired using a 3D Magnetization Prepared Rapid Gradient Echo (MPRAGE) sequence (repetition time (TR)=2300 ms, echo time (TE)=2.98, flip angle=9°, Field of View (FoV)=256×256 mm2, 176 slices, voxel size=1×1×1 mm3, pixelBandwidth=240 Hz/Px, acquisition time=5:51 mins). dMRI scans were acquired using an echo-planar imaging sequence (TR=9400 ms, TE=53 ms, flip angle=90°, gradient directions=30, b-value 1=0, b-value 2=1000, FoV=1152×1152 mm2, 31 slices, voxel size=2×2×2 mm3, acquisition time=6:41 mins).

Participants at The Ottawa Hospital were scanned on a Siemens 3.0 Tesla Trio Tim. T1 imaging was acquired using a 3D Magnetization Prepared Rapid Gradient Echo (MPRAGE) sequence (repetition time (TR)=2300 ms, echo time (TE)=2.98 ms, flip angle=9°, Field of View (FoV)=256×256 mm2, 176 slices, voxel size=1×1×1 mm3, pixelBandwidth=240 Hz/Px, acquisition time=5:51 mins). dMRI scans were acquired using an echo-planar imaging sequence (TR=9500 ms, TE=96 ms, flip angle=90°, gradient directions=30, b-value 1=0, b-value 2=1000, FoV=128×128 mm2, 2170 slices, voxel size=2×2×2 mm3, acquisition time=6:41 mins).

Calgary: MR images were acquired using a 3T MR scanner (Discovery MR750; GE Healthcare, Waukesha, WI) and a 12-channel, phased-array radiofrequency head coil. T1-weighted images were acquired for anatomical registration (3D inversion-prepared spoiled gradient echo: repetition time (TR)=7200 ms, echo time (TE)=23 ms, flip angle=10°, Field of View (FoV)=256×256 mm2, 176 slices, voxel size=1×1×1 mm3). dMRI scans were acquired using an echo-planar imaging sequence (TR=8000 ms, TE=66 ms, flip angle=90°, gradient directions=64, 77 slices, voxel size=2×2×2 mm3). One sequence with reversed phase encoding direction was acquired to correct for susceptibility induced distortions.

### 3.0 Data Transfer and Storage

Datasets from every included site were transferred to the advanced research computing platform provided by the Digital Research Alliance of Canada. This platform offers free storage allocations and computing resources for researchers across Canada. For this project, data are stored on an assigned allocation on the Graham servers that can be accessed remotely by members of the research team. Data were organized into a standardized format, known as the Brain Imaging Data Structure (BIDS) for storage and computational purposes. The conversion to the BIDS format is an automated process that organizes participant-level datasets into a unified structure that groups similar images together (e.g., anatomical images stored separately from diffusion images) and each image file is associated with a human- and machine-readable metadata file. Using a standardized file organization structure like BIDS, allows researchers to access and make use of free or open-source computational pipelines that require input data to conform to BIDS requirements.

### 4.0 Quality Control

Quality control (QC) was initiated at the participant level following preprocessing of T1w and DTI images. Two independent raters visually inspected T1w, DTI, and FA images to confirm appropriate co-registration and identify any artifacts or additional issues (e.g., significant warping in diffusion images or banding in T1w images). Based on this review, each rater assigned each participant's dataset a grade of “pass”, “fail”, or “unsure”. A decision of “fail” could result from an issue with any one of the images inspected. Ratings were then reviewed by postdoctoral fellow with expertise in diffusion MRI, and a final determination was made in instances where the two raters disagreed or were unsure. Depending on the nature of the issue identified, it was possible to re-preprocess the participant's dataset and undertake the QC process again with the re-preprocessed data, such as in the case of an issue with co-registering the DTI to the T1w image. However, participants that failed QC for reasons that could not be corrected (e.g., issues with the raw images) were excluded from further analyses. The percentage of participants failing QC at each site was as follows: Western (22.0%), MNI/QPN (10.5%), ONDRI (21.0%), and Calgary (15.6%).

### 5.0 MRI Data Processing

### 5.1 Pipeline Architecture

Snakebids, a workflow management system that handles neuroimaging data, was used to develop our pipeline. The pipeline takes advantage of parallelization for efficient task execution by utilizing unlimited cores for faster processing. Batch submission via Slurm is used for resource allocation and job management. The workflow itself consists of various rules, each representing a shell command or a Python script. Image processing tasks utilize tools such as Convert3D for manipulation of image data. Diffusion-based processing relies on MRtrix3 software. Surface-based image processing, involving CIFTI (Connectivity Informatics Technology Initiative; *.nii file extension) and GIFTI (Geometry format under the Neuroimaging Informatics Technology Initiative (NIfTI); *.gii file extension) data, relies on the functionality provided by the wb command tool from Connectome Workbench. Custom image processing can be seamlessly integrated using Python, particularly with the Nibabel package for reading and writing Nifti volumes. This allows for flexible and tailored image manipulation within the pipeline.

### 5.2 Data Preprocessing

To use the proposed pipeline, the data must be converted to the BIDS format. Once the data is in the BIDS format, the data goes through preprocessing. The preprocessing of the T1-weighted (T1w) data commences with skull-stripping using the synthstrip algorithm, followed by a bias field correction. T1w image processing was performed using FMRIB Software Library (FSL) 5.0.11 (https://fsl.fmrib.ox.ac.uk/fsl/fslwiki/) and Advanced Normalization Tools (ANTs) 2.2 (http://picsl.upenn.edu/software/ants). Brain Extraction based on nonlocal Segmentation Technique (BeaST) was used for skull-stripping T1w images 3T (https://github.com/khanlab/beast). Then bias fields for skull-stripped 3T T1w images were corrected using N4BiasFieldCorrection, followed by intensity normalization.

Subsequently, two alternative approaches are employed to obtain image segmentation of subcortical regions. These segmentations will be utilized to generate surfaces, facilitating surface-based tractography and parcellation. The first approach involves the utilization of a deep learning-based tool called SynthSeg for the segmentation of subcortical regions of interest. Following this, a template-shape-injection step is performed, employing the greedy algorithm for shape injection registration. This step involves injecting the template shape into the anatomical data of the subject. The preprocessed T1w images are registered to the standard space (MNI152NLin2009cAsym) and the CIT168 probabilistic subcortical atlas was used for single atlas-based segmentation (https://neurovault.org/collections/3145/). This high-resolution atlas clearly demarcates the outer boundaries of the striatum (i.e., caudate nucleus, putamen, nucleus accumbens), SNc, SNr, and VTA based on data from young controls in the Human Connectome Project database. Cortical labels were built from the Harvard-Oxford atlas, split into six regions in each hemisphere: limbic, caudal motor, rostral motor, executive, parietal and occipital.

To generate surface images from the segmented subcortical regions, several steps are involved. First, the c3d tool is employed to convert the template-based subcortical images into probability segmentations. Next, the pyvista library, a Python helper library for the Visualization Toolkit (VTK), was used to convert the segmentation into isosurfaces, which connect all points in a three-dimensional space. These isosurfaces in the template space are transformed to the T1w space of each subject using the -surface-apply-affine tool in Workbench. This will generate a surface image (.gii) of subcortical regions of interest to be used in surface-based tractography and parcellation.

Diffusion Weighted Imaging (DWI) preprocessing. All dMRI data was processed using containerized in-house applications, snakedwi and diffparc, which use the BIDS standards to perform systematized pre-processing, fitting, image registration, and tractography. In snakedwi, DWI data is preprocessed by denoising using dwidenoise, and correcting for ringing artifacts with mrdegibbs, which are two tools available in Mrtrix. Eddy current distortions were corrected using eddy (FSL). For datasets without multiple phase-encoding directions, we can perform a registration-based susceptibility distortion correction using greedy on the average b0 image using the T1w image as a reference. This registration process utilized synthesized T1w-contrast images obtained from the SynthSR tool to enhance the alignment between the b0 and T1w images. Snakedwi generates quality control reports that include overlays of visualizations illustrating the skull-stripping and registration, allowing for the identification of any potential failures in each participant. Registration of the final preprocessed diffusion-weighted image to the T1w space was also performed using SynthSR to produce images with matched contrast prior to registration.

In some patients and controls at Western. GRE sequences were acquired. GRE magnitude image collection from all echoes were averaged then skull-stripped using BeaST. Skull-stripped averaged GRE magnitude images were then linearly registered to the final postprocessed T1w images using FMRIB's Linear Image Registration Tool (FLIRT). An in-house singular value decomposition algorithm based on Walsh, Gmitro, and Marcellin (2000) was employed to reconstruct the GRE raw data.^{12,13} This algorithm gives the least squares best estimate of the magnetization and avoids phase singularities. QSM processing was performed as follows: spatial phase unwrapping was accomplished using a 3D best path algorithm. 14 The frequency at each voxel was then estimated by weighted least squares; each phase echo was weighted by the local signal-to-noise ratio in the corresponding T2*-weighted image. Finally, background removal and dipole inversion were performed simultaneously using a single-step QSM algorithm. 15 Since susceptibility values calculated by dipole inversion are relative with unknown offset, an offset was set by forcing the average value within the CSF to be zero parts per billion (ppb). Due to low contrast in QSM and R2* images, the transformation matrices from the earlier average GRE magnitude to T1w image registrations were used to perform linear registration of both quantitative maps onto T1w images using FLIRT. QSM images were then offset using the average susceptibility in the CSF of each participant as an internal reference, which allows for between subject comparisons to be performed.

### 5.3 Diffusion Tractography

The overall approach of the parcellation technique is to use diffusion tractography to obtain potential structural connections between a subcortical structure of interest and the cortex (or any specified set of targets), and to cluster the subcortical region into subregions based on its strength of connectivity to the targets. The pipeline leverages tools available in the MRtrix3 package to model diffusion data and obtain connectivity profiles. The software includes features such as constrained spherical deconvolution for estimating fibre orientation distributions and a probabilistic streamlines algorithm for white matter fibre tractography. Preprocessed diffusion-weighted image volumes in the T1w space were then used to fit tensors and estimate diffusion tensor metrics using mrtrix (74). The fibre orientation distribution (FOD) reconstruction using constrained spherical deconvolution (CSD) was performed by using an FA-based response function, with the number of spherical harmonics (l_max) chosen automatically based on the number of gradient directions. Tractography was performed from spheres of radius 0.5 mm centred at each vertex, using the iFOD2 algorithm.

### 5.4 Connectivity-Based Parcellation

Diffparc-surf uses probabilistic tractography with fODFs to connect the subcortical vertices, such as the striatum and VTASNc, to cortical targets. By default, the pipeline uses 250 streamlines (this is an adjustable parameter) produced by seeding from new random locations until the target number of streamlines is reached or the maximum number of seeds is exceeded. The tractography step employs iFOD2, a probabilistic algorithm that utilizes fibre orientation density (FODs) represented in the Spherical Harmonic (SH) basis. It generates candidate streamline paths by drawing short curved “arcs” and samples the underlying FOD amplitudes along these arcs using trilinear interpolation. Once the tracks are generated, the connectome of streamline count will be generated using the tck2connectome tool, which saves a table with the probability of connecting corresponding to seed and target. The connectivity data is then normalized by the mean value of a specific percentile (default is 95%). This normalization method is useful when there are outliers or extreme values in the dataset that can disproportionately affect the overall mean and standard deviation. By normalizing based on the percentile mean, the impact of outliers is reduced, and the data is rescaled to a more representative range. This data is eventually saved as a surface image (.gii) of the subcortical region, where the value of each vertex represents the streamline count for a specific target. A further smoothing step is applied to increase the signal-to-noise ratio of the images. Finally, the Workbench command -cifti-parcellate is used to parcellate the surface image into subregions, with each parcel consisting of vertices showing the maximum connectivity to a target of interest.

### 5.5 Feature Extraction

After the parcellation step is finished, the pipeline produces tabular data containing the features for each parcel across all subjects. The features are described below:

### 1. Connectivity of Parcels (Parcel_Conn):

This is a measure of connectivity from one brain region parcel to another brain structure.

2. FA and MD Along Pathways (bundleFA and bundleMD):

Streamline bundles that start from parcels (subregions) and reach the corresponding target are obtained by first finding the specific streamline that corresponds to the vertices labelled by that region (e.g., a bundle with streamlines from the Caudal motor striatal region that end up reaching the Caudal_motor cortical region). Then, the subset of those streamlines that reach the cortical target region is obtained using the tckedit command in MRtrix. The FA and MD values of the pathways are computed using the tensor2metric function found in MRtrix. The -fa option is used to obtain the FA metric, and -adc is used to obtain the mean apparent diffusion coefficient (ADC) of the diffusion tensor, often referred to as MD. These FA and MD maps can be used to mask the bundle tracks and calculate the FA and MD along pathways, referred to as bundleFA and bundleMD.

3. Surface Morphometry (inout):

After the initial rigid transformation is complete using greedy, a fluid transformation is applied using the same tool to calculate the displacement from the template to the subject image. The Workbench tool is then used to convert the warp fields of the transformation from itk to NIFTI “world” warpfields. Next, the transformation is applied to the surface using the -surface-apply-warpfield command to obtain the surface deformation. The surface-normals command is then used to generate the normal vectors of the surface as a metric file, which is smoothed using the -metric-smoothing command. Finally, the displacement is subtracted by the smoothed displacement using the -metric-math command in Workbench to obtain a spatially local displacement estimate. This is normalized by the average displacement vector in the local neighbourhood within 8 mm FWHM surf smoothing. To evaluate the displacement as an inward/outward displacement vector, the dot product with the surface normal is used as the surface morphometry feature inout.

4. Surface Area (surfarea) and Surface Area Ratio (surfarearatio):

The surface area of each parcel is calculated using the -surface-vertex-areas command in Workbench, which assigns one-third of the area of each triangle in the surface mesh in mm2. The surface area ratio, another surface morphometry feature similar to inout, is calculated using the log ratio of the subject surface area over the template surface area as a measure of expansion (+ve) or contraction (−ve) of the surface. Both subject and template surface areas are calculated using the same-surface-vertex-areas command, and the log ratio between them is calculated using-metric-math, also available in Workbench.

5. Surface Volume (surfvolmni):

To calculate the enclosed volume of the isosurfaces, pyvista was utilized to convert the surface gii into vtk polydata. This allowed us to accurately determine the volume enclosed by the surface and incorporate it into further analysis or modelling as needed. This feature is calculated within the whole subcortical region (per hemisphere) rather than within each parcel.

6. FA and MD on Surface (surfFA and surfMD):

FA and MD on the surfaces were calculated by converting the volumes of FA and MD maps into surfaces using-volume-to-surface-mapping in Workbench. Thresholding these surfaces by the subcortical parcellations, we can obtain surfFA and surfMD for each parcel.

### 7. Whole Brain Features:

Synth Seg is a deep learning-based tool for segmentation of brain scans of any contrast and resolution. This tool is available through the FreeSurfer package. We used synthseg in our pipeline to parcellate the whole cortex and also 32 regions (Default regions in FreeSurfer). Within each of these parcellation volumes (synthsegcortparc_volmni), FA and MD (synthsegcortparc_FA, synthhsegcortparc_MD) features were calculated.

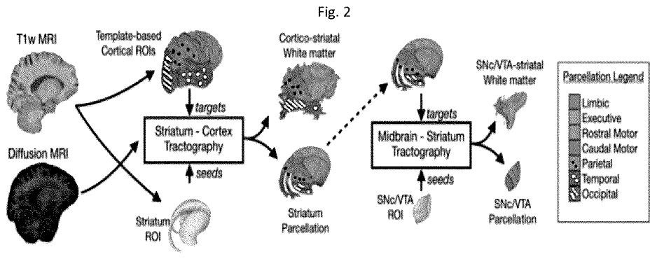

The parcellation of the striatum and VTA/SNc into subcortical regions are illustrated in FIG. 2. Striatum is segmented into caudal motor, rostral motor, parietal, executive, temporal, limbic, and occipital. The SNc/VTA are segmented into caudal motor SNc, rostral motor SNc, executive SNc, and limbic or VTA.

### 6.0 Planned Statistical Analyses/Modelling

We extracted a total of 458 features for each participant (Method 5.5 Feature Extraction) from subregions of subcortical and cortical areas, across both hemispheres. To investigate whether combining more homogeneous subregional measures of subcortical structures and cortex improves MRI potential for identifying NDs such as PD, we performed a series of supervised learning experiments. In these experiments, we evaluated the accuracy, sensitivity, and specificity of combining features from these more targeted subcortical subregions in classifying a) PD patients from healthy controls (Experiments 1, 2, 3), b) pre-clinical PD patients from healthy controls (Experiment 4), and c) pre-clinical PD patients from PD patients (Experiment 4), we used a variety of supervised, machine learning approaches (e.g., deep neural networks). Using machine learning techniques, inputs, here MRI features, are mapped to outputs, which, in this case, consist of the labels of a) PD or healthy age-matched controls (Experiments 1, 2, 3), or b) pre-clinical PD (i.e., RBD) or healthy age-matched controls (Experiment 4).

Experiments 1A and 1B: We evaluated the ability of XGBoost modelling classification to distinguish PD patients from healthy controls in almost our entire dataset. For this experiment, we used a wide age range (age 45 to 80) and a long duration of disease cut off (248 months) to use the majority of cases from our dataset. The purpose of this experiment was to determine if XGBoost modelling can successfully classify participants as PD or healthy control is a large, relatively heterogenous set of data. In Experiment 1A, XGBoost was given all brain features computed by the pipeline to create optimal models to classify PD and controls. In Experiment 1B, we removed the segmented subcortical brain features from the model development to assess its importance in creating models that successfully classify PDs and healthy controls.

Experiments 2A, 2B, 3A, and 3B: We employed an ensemble deep neural network (NN) approach to develop diagnostic models of early PD. This is a machine learning technique that combines multiple neural networks to improve predictive performance and robustness, in the development of diagnostic models. In Experiments 2 and 3, PD patients within 12 (n=66) and 24 (n=84) months of disease duration, respectively, were contrasted with healthy age-matched controls (n=142). In each experiment, we used Bagging (Bootstrap Aggregating), in which multiple neural networks were trained independently on different subsets of the training data, usually through bootstrapping (random sampling with replacement). The predictions of each network are then averaged, to make the final prediction. Bagging helps reduce overfitting and increases model stability.

Experiments 2A and 2B were conducted with a NN consisting of 2 layers, with 60 and 10 neurons in the first and second layers, respectively. Experiments 3A and 3B were conducted with a NN consisting of 2 layers, with 70 and 10 neurons in the first and second layers, respectively. In each experiment, we performed the following procedure thrice: 1) We randomly split our data into 80% for training and 20% for model testing. 2) Within our 80% training set, we performed k-fold cross validation, in which k=3. 3) Through 5 iterations, two folds were randomly selected for training and the third fold was used for cross-validation. 3) At each iteration, the classification performance (PD vs. healthy controls (HC)) of the optimized model, was tested in the 20% hold-out set. In this way, for 3 separate models, we obtained the average ROC/AUC, sensitivity, and specificity based on 5 train-validate-test iterations.

In Experiments 2A and 3A, we included all 458 features. Experiments 2B and 3B were identical to Experiments 2A and 3A in all respects, save for the fact that we included only cortical features and the total striatum, SNc/VTA, but no subcortical subregional measures. The rationale for this experiment was the same as for Experiment 1B—to assess the importance of segmented subcortical features in successful model development.

Experiment 4: We evaluated the ROC/AUC, sensitivity, and specificity of iron estimated with QSM in the SNc subregion in classifying 21 RBD patients (i.e., pre-clinical PD patients) from HCs and PD from healthy controls. Binary logistic regressions were conducted using the bilateral means of QSM, an estimate of iron from the SNc, to perform separate one-versus-one classifications: RBD versus HC, PD versus HC. The predicted probabilities from these regressions underwent 10 repeated k-folds cross validation and the ROC curves were plotted. The rationale for this experiment was to investigate whether our diagnostic method, combined with iron measures in the SNc, can correctly identify pre-clinical PD patients.

### Results

### Patients

There was a total of 581 participants in this study, 342 PD patients and 239 healthy age-matched controls, from Western, MNI, ONDRI, and Calgary. Table 3 presents the demographic and clinical details for participants in a) Experiment 1A/B, age range 45-80, PD patients≤248 months versus healthy age-matched controls, b) Experiment 2A/B age range 50-75, PD patients≤12 months since date of diagnosis (i.e., disease duration) versus healthy age-matched controls, c) Experiment 3A/B age range 50-75, PD patients≤24 months since date of diagnosis (i.e., disease duration) versus healthy age-matched controls PD patients≤24 disease duration. In Experiment 4 we compared RBD patients (from a separate dataset) with healthy controls. RBD

Experiment 1A: This section introduces a single XGBoost model that has been optimized to classify PD and age-matched healthy normal controls. It will serve as a reference point in a subsequent section where we aim to clarify performance variations across different training and test set samplings. All the machine-learning experiments we report here were conducted within a Python environment. However, missing data was imputed using the Multiple Imputation by Chained Equations (MICE) library within the R environment.

The model was trained using a set of 72 raw features, which were selected via a random forest classifier applied to the training dataset. The complete training dataset originally contained 460 features. No preprocessing steps were applied to the features. For this analysis, the study participants were limited to those aged between 45 and 80 years, with a maximum disease duration of 248 months.

Under these conditions, the dataset consisted of 537 cases, which were divided into training and test sets using an 80:20 split ratio. The random seed employed for data splitting was set to a predetermined value of two. During the training process, a 5-fold cross-validation approach was employed, coupled with automated hyperparameter tuning facilitated by the Optuna package (www.optuna.org).

Optuna uses a form of Bayesian optimization for parameter tuning. The core of Bayesian optimization in Optuna is a probabilistic model called Tree-structured Parzen Estimator (TPE). The TPE model is used to predict the objective value (e.g., AUC, accuracy, logloss) of a trial given its hyperparameters, and Optuna uses this model to suggest new sets of hyperparameters. In the context of this experiment, the range of hyperparameters was initially constrained manually and subsequently optimized over 100 trials using Optuna.

After the completion of the Optuna search trials, the best hyperparameters were extracted and then used to fit the final XGBoost model. To ensure consistency and reproducibility, all associated random seeds were held constant. The key performance metrics were obtained through application of the model, developed in the training set, to the hold-out test set.

We produced confusion matrices for each model as part of our standard workflow. These matrices were generated using both the default decision threshold and a threshold that maximizes the F1 Score.

Cross-validation performance metrics were generated for each model (i.e., training) and two variations of bootstrap resampling. In the first approach, the test predictions were resampled 500 times. In the second approach, the training set was resampled and assessed against the test sets 500 times after the initial model fitting and data split. These resampling techniques provided additional insights into the stability of the models, allowing us to assess variability using measures such as standard deviation, minimum, maximum, and a 95% confidence interval.

Finally, the feature importance plot was generated by the XGBoost default ‘weight’ method, which counts the number of times each feature is used to split the data across all trees in the ensemble. Features that are frequently used for splitting are considered more important.

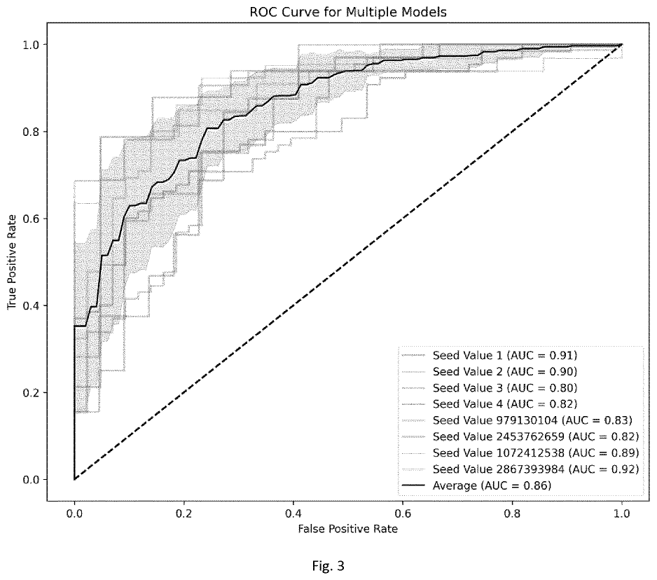

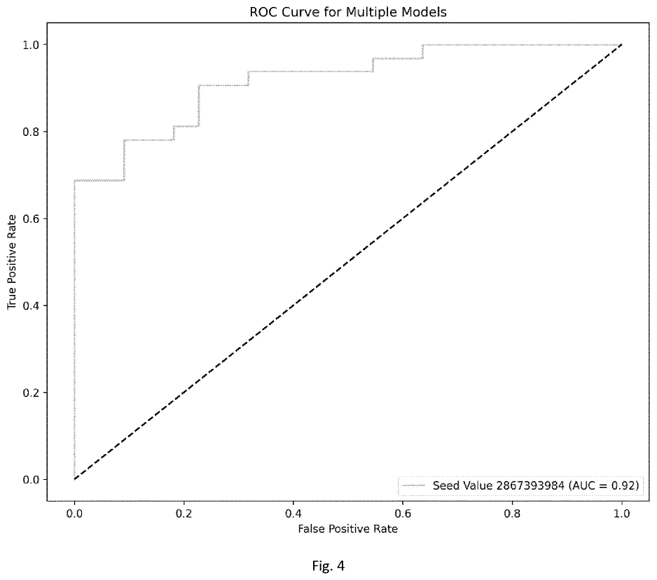

FIG. 3 demonstrates the AUCs calculated for eight experiments using eight different splits of the dataset into training and test sets. The splits were performed using a split seed variable. The split seeds used were 1, 2, 3, 4, and four randomly generated numbers. The seed number is fed into an algorithm that splits the dataset into a training set (80% of the dataset) and a test set (20% of the dataset). The AUCs ranged from 0.80 to 0.92 with an average of 0.86. FIG. 4 showed the AUC from the top performing model with an AUC of 0.92.

Experiment 1B: We repeated Experiment 1A under the same conditions with the same parameters, but with a truncated feature list. To evaluate the contribution of the segmented subcortical brain regions in the classification of PD and healthy controls, we removed them from the feature list and repeated the XGBoost modelling.

Similar to Experiment 1A, a total of eight trials using eight different data splits were performed. We used the same split seeds as used in Experiment 1A to allow for a direct comparison. XGBoost (see methods) was used to calculate AUCs, sensitivities, and specificities. Using all brain features to create models to distinguish PD from healthy controls, the mean AUC was 86, mean sensitivity 0.8769, and mean specificity 0.7180 (Experiment 1A). Using the same data splits, the same experiments were performed without the segmented subcortical regions contributing to model development. The performance of the models in correctly classifying cases as PDs or healthy controls decreased without the contribution of segmented subcortical regions. The average AUC decreased from 86 to 79.375 (p<0.05) and the average specificity from 0.7180 to 0.5872 (p<0.05). The change in sensitivity was not statistically significant (Table 4).

Experiments 2A-3B: In Experiments 2A and 3A, we included all 458 features. In Experiments 2B and 3B, we included only cortical features and total striatum, SNc/VTA but no subcortical subregional measures.

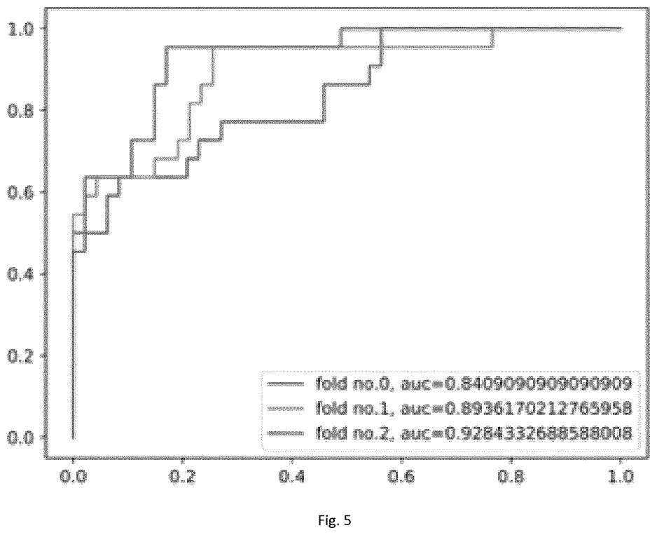

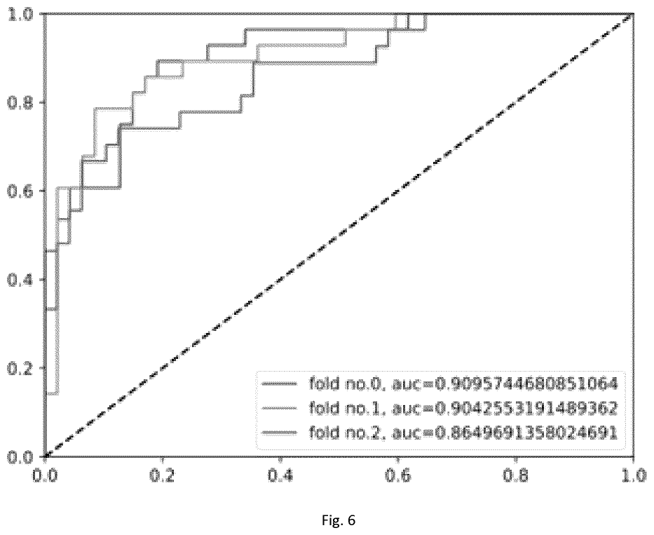

In Experiments 2A/B and 3A/B, PD patients within 12 months (n=66, Age: 64.09±4.53, M/F: 45/21) and 24 months (n=84; Age: 63.90±4.58, M/F: 56/27) disease duration, respectively, were contrasted with healthy age-matched controls (n=142 and Age: 62.67±4.82, M/F: 49/93). For Experiment 2A (i.e., PD=12 months disease duration), the average ROC/AUC of Models 1, 2, and 3 were 0.84, 0.89, and 0.93, respectively, for 5 classification evaluations on independent data (FIG. 5). Sensitivity and specificity were 86.4% and 85.1%. For Experiment 3A (i.e., PD=24 months disease duration), the average ROC/AUC of Models 1, 2, and 3 were 0.91, 0.90, and 0.86, respectively, for 5 classification evaluations on independent data (FIG. 6). Sensitivity and specificity of 89.2% and 80.8%. When segmented subcortical features are excluded, in the 12-month model, the AUC drops to 77.5% and the specificity drops to 43% (data not shown).

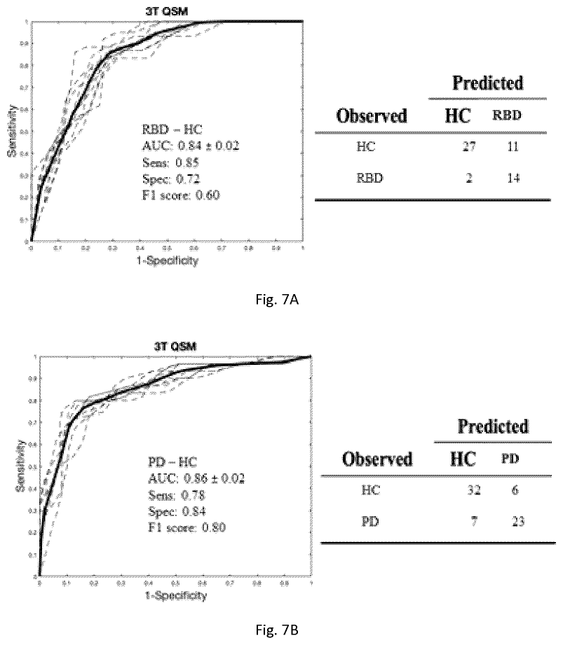

Experiment 4: Contrasting QSM in SNc at 3T, we found lower QSM (i.e., iron) in RBD versus HC with mean AUC=0.84, SEM=0.006, 95% CI=0.83-0.85, p<0.001, with a sensitivity of 0.85, specificity of 0.72 (FIG. 7A). Contrasting PD versus HC, the mean AUC was 0.86, SEM=0.005, 95% CI=0.85-0.88, p<0.001, with a sensitivity of 0.78, specificity of 0.84, and F1 score of 0.80 (FIG. 7B).

### Discussion

Here were present an MRI-based segmentation and classification pipeline that is fully automated from end-to-end. Our aim is to have a cloud-based system where standard MRI images can be uploaded and within minutes, a diagnosis can be obtained. Our methods are entirely specified objectively, and hence are completely reproducible. They do not require any imaging expertise. By segmenting subcortical structures into subregions that are more homogeneous functionally and with respect to their vulnerability to NDs, we have improved the accuracy, sensitivity, and specificity of structural MRI to diagnose a ND such as PD. PD currently lacks objective diagnostic tests as well as measures of progression and/or subtyping. Our results are based on large multi-centred samples of PD patients and healthy age-matched controls. In a heterogeneous group of PD patients, with a wide range of disease duration, relative to healthy age-matched controls, our method yields classification models that are sensitive, specific, and highly stable. We have not only presented our best results, but also average results from multiple experiments. These performance metrics, relying on automated MRI analyses, are within the range of diagnostic decisions of movement disorder neurologists who are in short supply and who generally require repeated clinical evaluations to achieve these levels of accuracy.

In Experiments 2A and 3A, even at 12-month and 24-months of disease duration, our PD-HC models revealed excellent diagnostic accuracy. Further, our models are highly stable and reliable. For our best model at 12 months (FIG. 5), classification accuracy was 93%. Sensitivity and specificity were 86.4% and 85.1%, respectively for this model. Though our models were developed entirely through machine learning methods (i.e., bottom up), the top features in these models were as predicted based on our understanding of the pathophysiology of PD (e.g., caudal motor, parietal subregions of striatum and SNc/VTA).

In Experiments 1B. 2B, and 3B, we found that removing features that relate to subregional measures of subcortical structures from our model, the performance deteriorates significantly (approximately 10 points), suggesting that this aspect of our approach of the present disclosure, gives rise to the accuracy, sensitivity, and specificity of our method.

Finally, in Experiment 4, we showed that our method is sufficiently sensitive to classify patients with pre-clinical/prodromal forms of PD, though our sample was small. This study showed that iron measures, coupled with our parcellation approach, suggest significant promise in single-subject level classification of patients even before symptoms of PD emerge.

### SUMMARY

Here, we present an automated system, based on a unique subcortical segmentation protocol, that can accurately diagnose PD using MRI and machine learning methods alone. We have tested our method on a large multi-centred dataset to demonstrate that it is effective in correctly classifying PD patients from healthy age-matched controls over a wide range of ages and disease durations. We have also demonstrated that it is effective in diagnosing early PD by performing experiments on cases restricted to 12-month and 24-month durations of illness. In combination with iron measures, we have even shown that it can identify early preclinical cases of PD (RBD patients). A very large literature investigating neuroimaging approaches to diagnosing, staging, and sub-typing PD patients, suggests that classification of patients from healthy controls is the most challenging achievement, as methods that distinguish PD patients from PD mimics with high accuracy, perform at nearly chance when contrasting PD patients versus healthy age-matched controls. 16 This owes to the fact that brain changes related to PD mimics are quite distinct and can often be seen with the naked eye. In contrast, at all stages of PD, structural MRI is entirely normal even when assessed with standard approaches by neuroradiologists with specialized training. Furthermore, our approach focusses on subcortex, yet parcellates and quantifies the entire brain in relation to subregions of these structures. It gives rise to many discrete measures (i.e., 458 in the current instantiation) that can be combined using machine learning techniques to track even small changes that might occur due to disease progression and/or unique sub-types/symptoms of PD. This will not only assist in managing PD and other NDs but could provide insights into disease pathophysiology, as well as sensitive and specific endpoints for testing definitive therapies. Subcortical brain regions are frequently the most affected areas in NDs. There is significant heterogeneity within these structures but segments that have different functions and involvement/susceptibility to NDs are not distinguishable on neuroimaging. Subcortical structures lack internal margins visible on neuroimaging to distinguish discrete segments. Consequently, standard neuroimaging is often uninformative in terms of diagnosis, progression, sub-typing, and prognostication of PD and other NDs. The proposed innovation is expected to greatly enhance the functionality of MRI in the management of NDs. Finally, given automation, our technique can be applied broadly by users who have no special expertise in imaging or in movement disorders/NDs. This is important given the lack of specialists to manage NDs, which is only worsening because these diseases are increasing in prevalence due to their association with aging and ongoing demographic changes in most of the world.

### Tables

TABLE 1
Summary of neuroimaging biomarker
research in Parkinson's Disease

| | |
|---|---|
| PD vs. Healthy Controls | PD vs. PD Mimics |

GROUP LEVEL ANALYSIS ONLY

| | |
|---|---|
| STRUCTURAL MRI | STRUCTURAL MRI |
| Hu et al. 2023 | Mitchell et al. 2022 |
| Sampedro et al. 2019 | IRON STUDIES |
| Du et al. 2012 | Meijer et al. 2015 |
| Mak et al. 2015 | Sjöström et al. 2017 |
| Mitchell et al. 2019 | PET STUDIES |
| Ofori et al. 2015 | Granert et al. 2015 |
| Schwarz et al. 2013 | Honkanen et al. 2019 |
| Tinaz et al. 2010 | Saari et al. 2017 |
| Zhou et al. 2021 | Shimada et al. 2009 |
| Rolheiser et al. 2011 | SPECT STUDIES |
| IRON STUDIES | Honkanen et al. 2019 |
| Du et al. 2012 | Saari et al. 2017 |
| Biondetti et al. 2020 | Shimada et al. 2009 |
| De Marzi et al. 2016 | |
| Du et al. 2015 | |
| Du et al. 2018 | |
| PET STUDIES | |
| Depierreux et al. 2021 | |
| Granert et al. 2015 | |
| Horsager et al. 2020 | |
| Nandhagopal et al. 2011 | |
| Holtbernd et al. 2015 | |
| Lee et al. 2000 | |
| Politis et al. 2010 | |
| Schindlbeck et al. 2020 | |
| Shang et al. 2021 | |
| SPECT STUDIES | |
| Lee et al. 2000 | |
| Nandhagopal et al. 2011 | |
| Politis et al. 2010 | |
| Schindlbeck et al. 2020 | |
| Sampedro et al. 2019 | |

CLASSIFICATION MODEL WITHOUT INDEPENDENT TESTING

| | | |
|---|---|---|
| No Independent | STRUCTURAL MRI | STRUCTURAL MRI |
| Validation | Nicoletti et al. 2008 | Mitchell et al. 2019 |
| | Prodoehl et al. 2013 | Nicoletti et al. 2008 |
| | Taniguchi et al. 2018 | Prodoehl et al. 2013 |
| | Vaillancourt et al. 2009 | Taniguchi et al. 2018 |
| | Zorzenoni et al. 2021 | IRON STUDIES |
| | IRON STUDIES | Mazzucchi et al. 2022 |
| | Bae et al. 2016 | Ohtsuka et al. 2014 |
| | Meijer et al. 2015 | Sjöström et al. 2017 |
| | Isaias et al. 2016 | Taniguchi et al. 2018 |
| | Noh et al. 2015 | PET STUDIES |
| | Ohtsuka et al. 2013 | Antonini et al. 1997 |
| | Ohtsuka et al. 2014 | Hellwig et al. 2012 |
| | Taniguchi et al. 2018 | SPECT STUDIES |
| | Ariz et al. 2023 | Hellwig et al. 2012 |
| | He et al. 2021 | |
| | Jokar et al. 2023 | |
| | Zhang et al. 2022 | |
| | PET STUDIES | |
| | Antonini et al. 1997 | |
| | Lin et al. 2014 | |
| | Tang et al. 2010 | |
| | SPECT STUDIES | |
| | Lin et al. 2014 | |
| | Ohtsuka et al. 2014 | |
| | Tang et al. 2010 | |
| k-folds Cross- | STRUCTURAL MRI | |
| Validation or | Adeli et al. 2016 | |
| Leave One Out | Amoroso et al. 2018 | |
| | Chakraborty et al. 2020 | |
| | Yang et al. 2021 | |
| | Zhao et al. 2022 | |
| | IRON STUDIES | |
| | Ariz et al. 2019 | |
| | Seong et al. 2023 | |

CLASSIFICATION MODEL WITH INDEPENDENT TESTING

| | | |
|---|---|---|
| Single Centre | | STRUCTURAL MRI |
| | | Chougar et al. 2021 |
| | | Mangesius et al. 2018 |
| Multicentre | STRUCTURAL MRI | STRUCTURAL MRI |
| | Camacho et al. 2023 | Archer et al. 2019 |
| | IRON STUDIES | |
| | Wang et al. 2023 | |

TABLE 2
Neuroimaging studies of Parkinson's Disease: Classification at the single subject level

| | | | | | | PD sample | | |
|---|---|---|---|---|---|---|---|---|
| | | | | | Automated | size | | Segmented |
| Author, Year, | | | | Independent | Image | greater | Imaging | subcortical |
| DOI | AUC | Sensitivity | Specificity | Validation? | Analysis? | than 100? | Alone? | subregions? |
| Adeli et al, 2016, | 0.85 to | ~0.85 | | No | Semi | Yes | Yes | No |
| doi.org/10.1016/j.neuroimage.2016.05.054 | 0.90 | | | | | | | |
| Amoroso et al, 2018, | 0.97 | 0.93 | 0.92 | No | Yes | Yes | No | No |
| doi.org/10.1016/j.media.2018.05.004 | | | | | | | | |
| Ariz et al, 2019, | 0.842 | 0.80 | 0.80 | Yes | Yes | No | Yes | Yes |
| doi.org/10.1109/TMI.2018.2872852 | | | | | | | | |
| Ariz et al, 2023, | 0.947 | | | No | Yes | No | Yes | Yes |
| doi.org/10.1101/2023.04.13.23288519 | | | | | | | | |
| Camacho et al, 2023, | 0.87 | 0.78 | 0.81 | Yes | Semi | Yes | Yes | No |
| doi.org/10.1016/j.nicl.2023.103405 | | | | | | | | |
| Chakraborty et al, 2020, | 0.98 | 0.943 | 0.943 | No | Semi | Yes | Yes | No |
| doi.org/10.3390/diagnostics10060402 | | | | | | | | |
| He et al, 2021, | 0.97 to | 0.90 to | 0.98 | No | No | No | No | Yes |
| doi.org/10.1016/j.neuroimage.2021.117810 | 0.98 | 0.93 | | | | | | |
| Hu et al, 2023, | | | | No | No | No | Yes | Yes |
| doi.org/10.1007/s00330-023-09780-0 | | | | | | | | |
| Jokar et al, 2023 | 0.947 | 0.88 | 0.91 | No | No | No | Yes | Yes |
| doi.org/10.1016/j.neuroimage.2022.119814 | | | | | | | | |
| Mitchell et al, 2022, | | | | NA | No | No | No | Yes |
| doi.org/10.1016/j.nicl.2022.103022 | | | | | | | | |
| Sampedro et al, 2019, | | | | NA | No | No | No | No |
| doi.org/10.1016/j.parkreldis.2019.09.031 | | | | | | | | |
| Sampedro et al, 2019, | | | | NA | No | No | No | No |
| doi.org/10.1016/j.nbd.2018.11.001 | | | | | | | | |
| Seong et al, 2023, | 0.994 | 0.98 | 0.94 | No | No | No | Yes | Yes |
| doi.org/10.1186/s12880-023-01018-1 | | | | | | | | |
| Shinde et al, 2019, | 0.913 | 0.86 | 0.70 | Yes | Yes | No | Yes | Yes |
| doi.org/10.1016/j.nicl.2019.101748 | | | | | | | | |
| Talai et al, 2021, | 0.69 to | | | No | Yes | No | Yes | No |
| doi.org/10.3389/fneur.2021.648548 | 0.99 | | | | | | | |
| Wang et al, 2023, | 0.845 | 0.771 | 0.806 | Yes | Yes | Yes | Yes | No |
| doi.org/10.1002/hbm.26399 | | | | | | | | |
| Yang et al, 2021 | 0.97 | 0.95 | | Yes | Yes | No | No | No |
| doi.org/10.1016/j.jneumeth.2020.109019 | | | | | | | | |
| Zhang et al, 2022, | 0.965 | 0.923 | 0.903 | No | No | No | No | Yes |
| doi.org/10.3233/JPD-223499 | | | | | | | | |
| Zhao et al, 2022, | 0.941 | | | Yes | Semi | Yes | Yes | No |
| doi.org/10.1007/s11682-022-00631-y | | | | | | | | |
| Zorzenon et al, 2021 | | 0.73 to | 0.47 to | No | No | No | Yes | Yes |
| doi.org/10.1016/j.parkreldis.2020.12.060 | | 1.0 | 0.93 | | | | | |

TABLE 3
Demographic information of study participants

| | PD | | | Controls | | |
|---|---|---|---|---|---|---|
| Site | Female | Male | Total | Female | Male | Total |

Dataset restricted to age 45-80, disease duration ≤248 months

| | | | | | | | | |
|---|---|---|---|---|---|---|---|---|
| UWO | N | 15 | 36 | 51 | 32 | 25 | 57 | |
| | Average age (y) | 64.3 | 68.8 | 67.5 | 63.3 | 63.3 | 63.3 | |
| | Average disease | 30.9 | 22.8 | 25.2 | N/A | N/A | N/A | |
| | duration (mo) | | | | | | | |
| MNI | N | 35 | 72 | 107 | 21 | 10 | 31 | |
| | Average age (y) | 62.4 | 63.8 | 63.4 | 63.8 | 67.9 | 65.1 | |
| | Average disease | 59.3 | 56.7 | 57.6 | N/A | N/A | N/A | |
| | duration (mo) | | | | | | | |
| ONDRI | N | 34 | 125 | 159 | 44 | 52 | 96 | |
| | Average age (y) | 67.5 | 67.4 | 67.4 | 64.8 | 69.6 | 67.4 | |
| | Average disease | 36.9 | 41.9 | 40.8 | N/A | N/A | N/A | |
| | duration (mo) | | | | | | | |
| Calgary | N | 19 | 26 | 45 | 19 | 12 | 31 | |
| | Average age (y) | 69.2 | 69.4 | 69.3 | 66.6 | 69.6 | 67.8 | |
| | Average disease | 74.3 | 50.0 | 60.3 | N/A | N/A | N/A | |
| | duration (mo) | | | | | | | |
| Total N | | 103 | 259 | 362 | 116 | 99 | 215 | |
| | | | | 362 | | | 215 | 577 |

Dataset restricted to age 50-75, disease duration ≤12 months

| | | | | | | | | |
|---|---|---|---|---|---|---|---|---|
| UWO | N | 6 | 20 | 26 | 31 | 19 | 50 | |
| | Average age (y) | 63.3 | 67.0 | 66.2 | 63.8 | 62.3 | 63.2 | |
| | Average disease | 3.75 | 2.60 | 2.87 | N/A | N/A | N/A | |
| | duration (mo) | | | | | | | |
| MNI | N | 8 | 13 | 21 | 19 | 7 | 26 | |
| | Average age (y) | 61.5 | 64.8 | 63.5 | 63.9 | 63.7 | 63.8 | |
| | Average disease | 7.50 | 7.77 | 7.67 | N/A | N/A | N/A | |
| | duration (mo) | | | | | | | |
| ONDRI | N | 10 | 29 | 39 | 41 | 41 | 82 | |
| | Average age (y) | 68.0 | 67.1 | 67.3 | 63.8 | 67.6 | 65.7 | |
| | Average disease | 0.50 | 0.50 | 0.50 | N/A | N/A | N/A | |
| | duration (mo) | | | | | | | |
| Calgary | N | 1 | 1 | 2 | 17 | 11 | 28 | |
| | Average age (y) | 67.9 | 70.4 | 69.2 | 65.3 | 68.9 | 66.8 | |
| | Average disease | 10.99 | 8.09 | 9.54 | N/A | N/A | N/A | |
| | duration (mo) | | | | | | | |
| Total N | | 25 | 63 | 88 | 108 | 78 | 186 | |
| | | | | 88 | | | 186 | 274 |

Dataset restricted to age 50-75, disease duration ≤24 months

| | | | | | | | | |
|---|---|---|---|---|---|---|---|---|
| UWO | N | 9 | 23 | 32 | 31 | 19 | 50 | |
| | Average age (y) | 62.8 | 67.3 | 66.1 | 63.8 | 62.3 | 63.2 | |
| | Average disease | 9.06 | 5.09 | 6.20 | NA | NA | NA | |
| | duration (mo) | | | | | | | |
| MNI | N | 12 | 20 | 32 | 19 | 7 | 26 | |
| | Average age (y) | 61.7 | 64.6 | 63.5 | 63.9 | 63.7 | 63.8 | |
| | Average disease | 13.00 | 13.45 | 13.28 | NA | NA | NA | |
| | duration (mo) | | | | | | | |
| ONDRI | N | 12 | 32 | 44 | 41 | 41 | 82 | |
| | Average age (y) | 68.5 | 67.3 | 67.6 | 63.8 | 67.6 | 65.7 | |
| | Average disease | 4.42 | 2.70 | 3.17 | NA | NA | NA | |
| | duration (mo) | | | | | | | |
| Calgary | N | 1 | 5 | 6 | 17 | 11 | 28 | |
| | Average age (y) | 67.9 | 70.2 | 69.8 | 65.3 | 68.9 | 66.8 | |
| | Average disease | 11.00 | 17.77 | 16.64 | NA | NA | NA | |
| | duration (mo) | | | | | | | |
| Total N | | 34 | 80 | 114 | 108 | 78 | 186 | |
| | | | | 114 | | | 186 | 300 |

TABLE 4
Repeat of Experiment 1a without inclusion
of segmented subcortical features

| | AUCs generated using | | | AUCs generated | | |
|---|---|---|---|---|---|---|
| | entire feature | | | without segmented | | |
| | list (Experiment 1a) | | | subcortical features | | |
| Seed | | Sensi- | Spec- | | Sensi- | Spec- |
| split* | AUC | tivity | ificity | AUC | tivity | ificity |
| 1 | 91 | 0.8769 | 0.6977 | 80 | 0.9231 | 0.5349 |
| 2 | 90 | 0.8462 | 0.7209 | 82 | 0.9231 | 0.6047 |
| 3 | 80 | 0.9385 | 0.6512 | 80 | 0.8769 | 0.5814 |
| 4 | 81 | 0.9077 | 0.6279 | 80 | 0.8000 | 0.7674 |
| 979130104 | 83 | 0.8615 | 0.7209 | 79 | 0.9692 | 0.5116 |
| 2453762659 | 82 | 0.8615 | 0.6744 | 79 | 0.9385 | 0.4884 |
| 1072412538 | 89 | 0.9077 | 0.8605 | 78 | 0.8462 | 0.6744 |
| 2867393984 | 92 | 0.8154 | 0.7907 | 77 | 0.8769 | 0.5349 |
| AVERAGE | 86 | 0.8769 | 0.7180 | 79.375 | 0.8942 | 0.5872 |

*The seed split variable is a variable used to split the data into a training set and a test set. Values 1, 2, 3, and 4 were used for the split algorithm, in addition to four randomly generated numbers.

### REFERENCES

1. Lang. A. E. & Lozano. A. M. Parkinson's disease. First of two parts. N. Engl. J. Med. 339, 1044-1053 (1998).
2. Goldman, J. G. & Postuma, R. Premotor and nonmotor features of Parkinson's disease. Curr. Opin. Neurol. 27, 434-441 (2014).
3. Majersik, J. J. et al. A Shortage of Neurologists We Must Act Now: A Report From the AAN 2019 Transforming Leaders Program. Neurology 10.1212/WNL.0000000000012111 (2021) doi: 10.1212/WNL.0000000000012111.
4. Rizzo, G. et al. Accuracy of clinical diagnosis of Parkinson disease: A systematic review and meta-analysis. Neurology 86, 566-576 (2016).
5. Virameteekul, S., Revesz, T., Jaunmuktane, Z., Warner, T. T. & De Pablo-Fernández, E. Clinical Diagnostic Accuracy of Parkinson's Disease: Where Do We Stand? Mov. Disord. Off. J. Mov. Disord. Soc. 38, 558-566 (2023).
6. Siderowf, A. et al. Assessment of heterogeneity among participants in the Parkinson's Progression Markers Initiative cohort using α-synuclein seed amplification: a cross-sectional study. Lancet Neurol. 22, 407-417 (2023).
7. Oftedal, L., Maple-Grødem, J., Tysnes, O.-B., Alves, G. & Lange, J. Seed Amplification Assay as a Diagnostic Tool in Newly-Diagnosed Parkinson's Disease. J. Park. Dis. 13, 841-844 (2023).
8. Prasuhn, J. et al. Clinical MR imaging in Parkinson's disease: How useful is the swallow tail sign? Brain Behav. 11, e02202 (2021).
9. Shams, S. et al. MRI of the Swallow Tail Sign: A Useful Marker in the Diagnosis of Lewy Body Dementia? AJNR Am. J. Neuroradiol. 38, 1737-1741 (2017).
10. Wang, N., Yang, H., Li, C., Fan, G. & Luo, X. Using ‘swallow-tail’ sign and putaminal hypointensity as biomarkers to distinguish multiple system atrophy from idiopathic Parkinson's disease: A susceptibility-weighted imaging study. Eur. Radiol. 27, 3174-3180 (2017).
11. Tziortzi, A. C. et al. Connectivity-based functional analysis of dopamine release in the striatum using diffusion-weighted MRI and positron emission tomography. Cereb. Cortex N. Y. N 1991 24, 1165-1177 (2014).
12. Walsh, D. O., Gmitro, A. F. & Marcellin, M. W. Adaptive reconstruction of phased array MR imagery. Magn Reson Med 43, 682-690 (2000).
13. Klassen, L. M. & Menon, R. S. Optimal phase sensitive combination of multi-channel, multi-echo images, in Proceedings of the 21st Annual Meeting of ISMRM, Salt Lake City, Utah (2013).
14. Abdul-Rahman, H. S. et al. Fast and robust three-dimensional best path phase unwrapping algorithm. Appl. Opt. 46, 6623-6635 (2007).
15. Chatnuntawech, I. et al. Single-step quantitative susceptibility mapping with variational penalties. NMR Biomed 30, (2017).
16. Archer, D. B. et al. Development and Validation of the Automated Imaging Differentiation in Parkinsonism (AID-P): A Multi-Site Machine Learning Study. Lancet Digit. Health 1, e222-e231 (2019).

Through the embodiments that are illustrated and described, the currently contemplated best mode of making and using the disclosure is described. Without further elaboration, it is believed that one of ordinary skill in the art can, based on the description presented herein, utilize the present disclosure to the full extent. All publications cited herein are incorporated by reference.

Although the description above contains many specificities, these should not be construed as limiting the scope of the disclosure, but as merely providing illustrations of some of the presently embodiments of this disclosure.

1. A transmission device comprising:
(i) a support frame;
(ii) a driven wheel supported for rotation relative to the support frame;
(iii) a drive wheel supported for rotation relative to the support frame such that a peripheral boundary of the drive wheel is in proximity to an engaged portion of the driven wheel, the drive wheel being configured to receive an input rotation, and the drive wheel being operatively connected to the driven wheel to transfer a rotation of the drive wheel in a first direction to a rotation of the driven wheel in a corresponding direction whereby a nip point is defined by a convergence between the peripheral boundary of the drive wheel and the engaged portion of the driven wheel as the drive wheel and the driven wheel are rotated in said first direction and said corresponding direction respectively;
(iv) a plurality of driven elements supported on the driven wheel at circumferentially spaced apart locations about the driven wheel, each driven element being supported for reciprocating movement between a first position in interference with the drive wheel at the nip point and a second position displaced radially of the driven wheel relative to the first position whereby each driven element is arranged to be driven from the first position to the second position as it passes through the nip point between the drive wheel and the driven wheel;
(v) a transfer linkage operatively connected between the driven elements and the driven wheel so as to transfer the reciprocating movement of the driven elements to rotation of the driven wheel in said corresponding direction; and
(vi) an output member driven by the transfer linkage to provide an output rotation.

2. The device according to claim 1 wherein the drive wheel is supported above the driven wheel such that the engaged portion of the driven wheel is in proximity to a top of the driven wheel and the driven elements are driven radially inward of the driven wheel from the first position to the second position.

3. The device according to claim 1 wherein the driven wheel is an annular body and wherein the drive wheel is supported within the annular body above a lower portion of the driven wheel such that the engaged portion of the driven wheel is an inner boundary of the annular body in proximity to the driven wheel and the driven elements are driven radially outward of the driven wheel from the first position to the second position.

4. The device according to claim 1 wherein the driven elements are supported on the driven wheel for sliding movement along a linear axis between the first position and the second position.

5. The device according to claim 4 wherein the linear axis of each driven element is oriented radially of the driven wheel.

6. The device according to claim 1 wherein each driven element includes a head surface arranged to be engaged by the drive wheel at the nip point, the head surface extending in said corresponding direction of rotation of the driven wheel at a slope radially of the driven wheel.

7. The device according to claim 1 wherein each driven element includes a head surface arranged to be engaged by the drive wheel at the nip point, each head surface being directly adjacent the head surface of each adjacent driven element in the circumferential direction.

8. The device according to claim 1 wherein the drive wheel is supported on the support frame for floating movement relative to the support frame whereby weight of the drive wheel is primarily carried by the engaged portion of the driven wheel upon which the drive wheel is seated.

9. The device according to claim 1 wherein the drive wheel is weighted so as to have a greater mass than the driven wheel.

10. The device according to claim 1 wherein a portion of the drive wheel makes rolling contact with a portion of the driven wheel.

11. The device according to claim 1 wherein the drive wheel includes a first gear formed thereon for rotation with the drive wheel, wherein the driven wheel includes a second gear formed thereon for rotation with the driven wheel, and wherein the first gear and the second gear are operatively connected to transfer rotation from the drive wheel to the driven wheel.

12. The device according to claim 1 further comprising a starter motor operatively connected to the drive wheel so as to provide the input rotation to the drive wheel.

13. The device according to claim 12 further comprising a reduction drive connecting the starter motor to the drive wheel such that the drive wheel is driven at a slower speed than an output speed of the starter motor.

14. The device according to claim 1 wherein the transfer linkage comprises:
a biasing element associated with each driven element in which the biasing element biases the associated driven element to return from the second position to the first position after passing through the nip point between the drive wheel and the driven wheel;
a driven gear associated with each driven element in which the driven gear is rotatably supported on the driven wheel;
a one-way drive arrangement associated with each driven element in which the one-way drive arrangement operatively connects the associated driven element to the associated driven gear so as to (a) drive the driven gear in a drive direction when the driven element is displaced from the first position to the second position, and (b) allow the driven gear to continue to rotate in the drive direction when the driven element is returned from the second position to the first position;
a transfer gear arranged to transfer drive from the driven gears in the drive direction to rotation of the driven wheel in said corresponding direction.

15. The device according to claim 14 wherein the biasing element of each driven element comprises a biasing spring connected under compression between the driven element and a respective mounting surface on the driven wheel.

16. The device according to claim 14 wherein each driven element is movable away from the second position beyond the first position to a respective overextended position, wherein the device further comprises a cushion spring associated with each driven element, and wherein each cushion spring is operatively connected to the associated driven element for biasing the associated driven element to the first position from the overextended position.

17. The device according to claim 14 wherein the transfer gear comprises a ring gear mounted in fixed relation to the support frame whereby meshing engagement of the rotating driven gears with the ring transfer drives rotation of the driven wheel relative to the transfer gear on the support frame.

18. The device according to claim 14 wherein the driven gear associated with each driven element comprises a first driven gear and a second driven gear connected for rotation together by a drive shaft and wherein the transfer gear comprises a first transfer gear operatively connected to the first driven gears and a second transfer gear operatively connected to the second driven gears.

19. The device according to claim 14 wherein each one-way drive arrangement comprises:
a row of gear teeth supported on the associated driven element;
a spur gear rotatably supported on the driven wheel in meshing engagement with the row of gear teeth of the associated driven element;
a drive shaft connecting the spur gear with the associated driven gear; and
a one-way clutch transferring drive in only one direction of movement, the one-way clutch being operatively connected: (i) between the driven element and the row of gear teeth, (ii) between the spur gear and the drive shaft, or (iii) between the drive shaft and the driven gear.

20. The device according to claim 1 wherein the transfer linkage comprises:
a crankshaft assembly supported for rotation relative to the support frame, the crankshaft assembly comprising (i) a plurality of crankpins and (ii) a plurality of connecting rods coupling the crankpins to the driven elements respectively to drive rotation of the crankshaft assembly responsive to reciprocation of the driven elements; and
at least one transfer gear driven to rotate by the crankshaft assembly, said at least one transfer gear being operatively connected to the driven wheel such that rotation of the crankshaft assembly drives rotation of the driven wheel relative to the support frame.
